SEQUENCE LISTING (59 sequences)

SEQ ID NO: 1 - DNA, 1322 bp, homo sapiens
tcctcaaaggaggggcagagcctgcgcagggcaggagcagctggcccactggcggcccgcaacactccgtctcaccctctgggcccactgcatctagaggagggccgtctgtgaggccactacccctccagcaactgggaggtgggactgtcagaagctggcccagggtggtggtcagctgggtcagggacctacggcacctgctggaccacctcgccttctccatcgaagcagggaagtgggagcctcgagccctcgggtggaagctgaccccaagccacccttcacctggacaggatgagagtgtcaggtgtgcttcgcctcctggccctcatctttgccatagtcacgacatggatgtttattcgaagctacatgagcttcagcatgaaaaccatccgtctgccacgctggctggcagcctcgcccaccaaggagatccaggttaaaaagtacaagtgtggcctcatcaagccctgcccagccaactactttgcgtttaaaatctgcagtggggccgccaacgtcgtgggccctactatgtgctttgaagaccgcatgatcatgagtcctgtgaaaaacaatgtgggcagaggcctaaacatcgccctggtgaatggaaccacgggagctgtgctgggacagaaggcatttgacatgtactctggagatgttatgcacctagtgaaattccttaaagaaattccggggggtgcactggtgctggtggcctcctacgacgatccagggaccaaaatgaacgatgaaagcaggaaactcttctctgacttggggagttcctacgcaaaacaactgggcttccgggacagctgggtcttcataggagccaaagacctcaggggtaaaagcccctttgagcagttcttaaagaacagcccagacacaaacaaatacgagggatggccagagctgctggagatggagggctgcatgcccccgaagccattttagggtggctgtggctcttcctcagccaggggcctgaagaagctcctgcctgacttaggagtcagagcccggcaggggctgaggaggaggagcagggggtgctgcgtggaaggtgctgcaggtccttgcacgctgtgtcgcgcctctcctcctcggaaacagaaccctcccacagcacatcctacccggaagaccagcctcagagggtccttctggaaccagctgtctgtggagagaatggggtgctttcgtcagggactgctgacggctggtcctgaggaaggacaaactgcccagacttgagcccaattaaattttatttttgctggttttgaatgaaaaaaaaaaaaaaa

SEQ ID NO: 2 - DNA, 1460 bp, homo sapiens
tgcagcgcaggtgagctctcctgaggacctctctgtcagctcccctgattgtagggaggatccagtgtggcaagaaactcctccagcccagcaagcagctcaggatgttcctgaaggccgtggtcctgaccctggccctggtggctgtcgccggagccagggctgaggtcagtgctgaccaggtggccacggtgatgtgggactacttcagccagctgagcaacaatgccaaggaggccgtggaacatctccagaaatctgaactcacccagcaactcaatgccctcttccaggacaaacttggagaagtgaacacttacgcaggtgacctgcagaagaagctggtgccctttgccaccgagctgcatgaacgcctggccaaggactcggagaaactgaaggaggagattgggaaggagctggaggagctgagggcccggctgctgccccatgccaatgaggtgagccagaagatcggggacaacctgcgagagcttcagcagcgcctggagccctacgcggaccagctgcgcacccaggtcagcacgcaggccgagcagctgcggcgccagctgaccccctacgcacagcgcatggagagagtgctgcgggagaacgccgacagcctgcaggcctcgctgaggccccacgccgacgagctcaaggccaagatcgaccagaacgtggaggagctcaagggacgccttacgccctacgctgacgaattcaaagtcaagattgaccagaccgtggaggagctgcgccgcagcctggctccctatgctcaggacacgcaggagaagctcaaccaccagcttgagggcctgaccttccagatgaagaagaacgccgaggagctcaaggccaggatctcggccagtgccgaggagctgcggcagaggctggcgcccttggccgaggacgtgcgtggcaacctgaggggcaacaccgaggggctgcagaagtcactggcagagctgggtgggcacctggaccagcaggtggaggagttccgacgccgggtggagccctacggggaaaacttcaacaaagccctggtgcagcagatggaacagctcaggcagaaactgggcccccatgcgggggacgtggaaggccacttgagcttcctggagaaggacctgagggacaaggtcaactccttcttcagcaccttcaaggagaaagagagccaggacaagactctctccctccctgagctggagcaacagcaggaacagcagcaggagcagcagcaggagcaggtgcagatgctggcccctttggagagctgagctgcccctggtgcactggccccaccctcgtggacacctgccctgccctgccacctgtctgtctgtctgtcccaaagaagttctggtatgaacttgaggacacatgtccagtgggaggtgagaccacctctcaatattcaataaagctgctgagaatctagcctc

SEQ ID NO: 3 - DNA, 753 bp, homo sapiens
gttgtggctgtggagcggaagtgggtctcaaccactataaatcctctctgtgcccgtccggagctggtgaggacagcctgccagagtctggtctctggacactatgggcacacgactcctcccagctctgtttcttgtcctcctggtattgggatttgaggtccaggggacccaacagccccagcaagatgagatgcctagcccgaccttcctcacccaggtgaaggaatctctctccagttactgggagtcagcaaagacagccgcccagaacctgtacgagaagacatacctgcccgctgtagatgagaaactcagggacttgtacagcaaaagcacagcagccatgagcacttacacaggcatttttactgaccaagttctttctgtgctgaagggagaggagtaacagccagaccccccatcagtggacaaggggagagtcccctactcccctgatcccccaggttcagactgagctcccccttcccagtagctcttgcatcctcctcccaactctagcctgaattcttttcaataaaaaatacaattcaagttgcttctcatggatggcactgcttttctgaggactcaagggccaagatggaggggctgactcagtccagccaacatttaatgagcacctactttatgtatggagctctaacccatgggtccatgggaataaagcagtgaatagtaacaataaataatcgtaacaaaaaaaaaaaaaaaaaaaaaaaaaaaaaaaaa

SEQ ID NO: 4 - DNA, 851 bp, homo sapiens
cacttcaaccggttgtcgccccagtggccgccctctgagcacgtgttactgccagtctgcgtcagcgttgggtaaatacatgactggccgacgccgccgggcggggctatttaagagacagccgcccgctggtcctccctgaacttggctcagctgccgggctgctccggttggaaacgccaagccagctgcgtcctaatccaaaagccatgaacagcggcgtgtgcctgtgcgtgctgatggcggtactggcggctggcgccctgacgcagccggtgcctcccgcagatcccgcgggctccgggctgcagcgggcagaggaggcgccccgtaggcagctgagggtatcgcagagaacggatggcgagtcccgagcgcacctgggcgccctgctggcaagatacatccagcaggcccggaaagctccttctggacgaatgtccatcgttaagaacctgcagaacctggaccccagccacaggataagtgaccgggactacatgggctggatggattttggccgtcgcagtgccgaggagtatgagtacccctcctagaggacccagccgccatcagcccaacgggaagcaacctcccaacccagaggaggcagaataagaaaacaatcacactcataactcattgtctgtggagtttgacattgtatgtatctatttattaagttctcaatgtgaaaaatgtgtctgtaagattgtccagtgcaaccacacacctcaccagaattgtgcaaatggaagacaaaatgttttcttcatctgtgactcctggtctgaaaatgttgttatgctattaaagtgatttcattctgaaaaaaaaaaaaaaaaaaaaaa

SEQ ID NO: 5 - DNA, 1886 bp, homo sapiens
cgcgcagcccctccggccgcgggcgcagcgggggcgctggtggagctgcgaagggccaggtccggcgggcggggcggcggctggcactggctccggactctgcccggccagggcggcggctccagccgggagggcgacgtggagcggccacgtggagcggcccgggggaggctggcggcgggaggcgaggcgcgggcggcgcagcagccaggagcgcccacggagctggacccccagagccgcgcggcgccgcagcagttccaggaaggatgttacctttgacgatgacagtgttaatcctgctgctgctccccacgggtcaggctgccccaaaggatggagtcacaaggccagactctgaagtgcagcatcagctcctgcccaaccccttccagccaggccaggagcagctcggacttctgcagagctacctaaagggactaggaaggacagaagtgcaactggagcatctgagccgggagcaggttctcctctacctctttgccctccatgactatgaccagagtggacagctggatggcctggagctgctgtccatgttgacagctgctctggcccctggagctgccaactctcctaccaccaacccggtgatattgatagtggacaaagtgctcgagacgcaggacctgaatggggatgggctcatgacccctgctgagctcatcaacttcccgggagtagccctcaggcacgtggagcccggagagccccttgctccatctcctcaggagccacaagctgttggaaggcagtccctattagctaaaagcccattaagacaagaaacacaggaagcccctggtcccagagaagaagcaaagggccaggtagaggccagaagggagtctttggatcctgtccaggagcctgggggccaggcagaggctgatggagatgttccagggcccagaggggaagctgagggccaggcagaggctaaaggagatgcccctgggcccagaggggaagctgggggccaggcagaggctgaaggagatgcccccgggcccagaggggaagctgggggccaggcagaggccagggagaatggagaggaggccaaggaacttccaggggaaacactggagtctaagaacacccaaaatgactttgaggtgcacattgttcaagtggagaatgatgagatctagatcttgaagatacaggtaccccacgaagtctcagtgccagaacataagccctgaagtgggcaggggaaatgtacgctgggacaaggaccatctctgtgccccctgtctggtcccagtaggtatcaggtctttctgtgcagctcagggagaccctaagttaaggggcagattaccaataaagaactgaatgaattcatccccccgggccacctctctacccgtccagcctgcccagaccctctcagaggaacggggttggggaccgaaaggacagggatgccgcctgcccagtgtttctgggcctcacggtgctccggcagcagagcgcatggtgctagccatggccggctgcagaggacccagtgaggaaagctcagtctatccctgggccccaaaccctcaccggttccccctcacctggtgttcagacaccccatgctctcctgcagctcagggcaggtgaccccatccccagtaatattaatcatcactagaactttttgagagccttgtacacatcaggcatcatgctgggcattttatatatgattttatcctcacaataattctgtagccaagcagaattggttccatttgacagatgaagaaattgaggcagattgcgttaagtgctgtaccctaaggtgatatgcagctaattaaatggcagatttgaaaaaaaaaaaaaaaaaaaaaaaaaaaaaaaaaaaa

SEQ ID NO: 6 - DNA, 2157 bp, homo sapiens
gctcccagccaagaacctcggggccgctgcgcggtggggaggagttccccgaaacccggccgctaagcgaggcctcctcctcccgcagatccgaacggcctgggcggggtcaccccggctgggacaagaagccgccgcctgcctgcccgggcccggggagggggctggggctggggccggaggcggggtgtgagtgggtgtgtgcggggggcggaggcttgatgcaatcccgataagaaatgctcgggtgtcttgggcacctacccgtggggcccgtaaggcgctactatataaggctgccggcccggagccgccgcgccgtcagagcaggagcgctgcgtccaggatctagggccacgaccatcccaacccggcactcacagccccgcagcgcatcccggtcgccgcccagcctcccgcacccccatcgccggagctgcgccgagagccccagggaggtgccatgcggagcgggtgtgtggtggtccacgtatggatcctggccggcctctggctggccgtggccgggcgccccctcgccttctcggacgcggggccccacgtgcactacggctggggcgaccccatccgcctgcggcacctgtacacctccggcccccacgggctctccagctgcttcctgcgcatccgtgccgacggcgtcgtggactgcgcgcggggccagagcgcgcacagtttgctggagatcaaggcagtcgctctgcggaccgtggccatcaagggcgtgcacagcgtgcggtacctctgcatgggcgccgacggcaagatgcaggggctgcttcagtactcggaggaagactgtgctttcgaggaggagatccgcccagatggctacaatgtgtaccgatccgagaagcaccgcctcccggtctccctgagcagtgccaaacagcggcagctgtacaagaacagaggctttcttccactctctcatttcctgcccatgctgcccatggtcccagaggagcctgaggacctcaggggccacttggaatctgacatgttctcttcgcccctggagaccgacagcatggacccatttgggcttgtcaccggactggaggccgtgaggagtcccagctttgagaagtaactgagaccatgcccgggcctcttcactgctgccaggggctgtggtacctgcagcgtgggggacgtgcttctacaagaacagtcctgagtccacgttctgtttagctttaggaagaaacatctagaagttgtacatattcagagttttccattggcagtgccagtttctagccaatagacttgtctgatcataacattgtaagcctgtagcttgcccagctgctgcctgggcccccattctgctccctcgaggttgctggacaagctgctgcactgtctcagttctgcttgaatacctccatcgatggggaactcacttcctttggaaaaattcttatgtcaagctgaaattctctaattttttctcatcacttccccaggagcagccagaagacaggcagtagttttaatttcaggaacaggtgatccactctgtaaaacagcaggtaaatttcactcaaccccatgtgggaattgatctatatctctacttccagggaccatttgcccttcccaaatccctccaggccagaactgactggagcaggcatggcccaccaggcttcaggagtaggggaagcctggagccccactccagccctgggacaacttgagaattccccctgaggccagttctgtcatggatgctgtcctgagaataacttgctgtcccggtgtcacctgcttccatctcccagcccaccagccctctgcccacctcacatgcctccccatggattggggcctcccaggccccccaccttatgtcaacctgcacttcttgttcaaaaatcaggaaaagaaaagatttgaagaccccaagtcttgtcaataacttgctgtgtggaagcagcgggggaagacctagaaccctttccccagcacttggttttccaacatgatatttatgagtaatttattttgatatgtacatctcttattttcttacattatttatgcccccaaattatatttatgtatgtaagtgaggtttgttttgtatattaaaatggagtttgtttgtaaaaaaaaaaaaaaaaaaaa

SEQ ID NO: 7 - DNA, 575 bp, homo sapiens
ggcactgctgccatgaatgccttcctgctctccgcactgtgcctccttggggcctgggccgccttggcaggaggggtcaccgtgcaggatggaaatttctccttttctctggagtcagtgaagaagctcaaagacctccaggagccccaggagcccagggttgggaaactcaggaactttgcacccatccctggtgaacctgtggttcccatcctctgtagcaacccgaactttccagaagaactcaagcctctctgcaaggagcccaatgcccaggagatacttcagaggctggaggaaatcgctgaggacccgggcacatgtgaaatctgtgcctacgctgcctgtaccggatgctaggggggcttgcccactgcctgcctcccctccgcagcagggaagctcttttctcctgcagaaagggccacccatgatactccactcccagcagctcaacctaccctggtccagtcgggaggagcagcccggggaggaactgggtgactggaggcctcgccccaacactgtccttccctgccacttcaacccccagctaataaaccagattccagagt

SEQ ID NO: 8 - DNA, 878 bp, homo sapiens
cagattttcaggttgattgatgtgggacagcagccacaatgaggaactcctatagatttctggcatcctctctctcagttgtcgtttctctcctgctaattcctgaagatgtctgtgaaaaaattattggaggaaatgaagtaactcctcattcaagaccctacatggtcctacttagtcttgacagaaaaaccatctgtgctggggctttgattgcaaaagactgggtgttgactgcagctcactgtaacttgaacaaaaggtcccaggtcattcttggggctcactcaataaccagggaagagccaacaaaacagataatgcttgttaagaaagagtttccctatccatgctatgacccagccacacgcgaaggtgaccttaaacttttacagctgacggaaaaagcaaaaattaacaaatatgtgactatccttcatctacctaaaaagggggatgatgtgaaaccaggaaccatgtgccaagttgcagggtgggggaggactcacaatagtgcatcttggtccgatactctgagagaagtcaatatcaccatcatagacagaaaagtctgcaatgatcgaaatcactataattttaaccctgtgattggaatgaatatggtttgtgctggaagcctccgaggtggaagagactcgtgcaatggagattctggaagccctttgttgtgcgagggtgttttccgaggggtcacttcctttggccttgaaaataaatgcggagaccctcgtgggcctggtgtctatattcttctctcaaagaaacacctcaactggataattatgactatcaagggagcagtttaaataaccgtttcctttcatttactgtggcttcttaatcttttcacaaataaa

SEQ ID NO: 9 - DNA, 1578 bp, homo sapiens
agtgtggtactttgtcttgaggagatgtcctggactcacacggaaacttagggctacggaatgaagttctcactcccattaggtgacaggtttttagagaagccaatcagcgtcgccgcggtcctggttctaaagtcctcgctcacccacccggactcattctccccagacgccaaggatggtggtcatggcgccccgaaccctcttcctgctgctctcgggggccctgaccctgaccgagacctgggcgggctcccactccatgaggtatttcagcgccgccgtgtcccggcccggccgcggggagccccgcttcatcgccatgggctacgtggacgacacgcagttcgtgcggttcgacagcgactcggcgtgtccgaggatggagccgcgggcgccgtgggtggagcaggaggggccggagtattgggaagaggagacacggaacaccaaggcccacgcacagactgacagaatgaacctgcagaccctgcgcggctactacaaccagagcgaggccagttctcacaccctccagtggatgattggctgcgacctggggtccgacggacgcctcctccgcgggtatgaacagtatgcctacgatggcaaggattacctcgccctgaacgaggacctgcgctcctggaccgcagcggacactgcggctcagatctccaagcgcaagtgtgaggcggccaatgtggctgaacaaaggagagcctacctggagggcacgtgcgtggagtggctccacagatacctggagaacgggaaggagatgctgcagcgcgcggacccccccaagacacacgtgacccaccaccctgtctttgactatgaggccaccctgaggtgctgggccctgggcttctaccctgcggagatcatactgacctggcagcgggatggggaggaccagacccaggacgtggagctcgtggagaccaggcctgcaggggatggaaccttccagaagtgggcagctgtggtggtgccttctggagaggagcagagatacacgtgccatgtgcagcatgaggggctgccggagcccctcatgctgagatggaagcagtcttccctgcccaccatccccatcatgggtatcgttgctggcctggttgtccttgcagctgtagtcactggagctgcggtcgctgctgtgctgtggagaaagaagagctcagattgaaaaggagggagctactctcaggctgcaatgtgaaacagctgccctgtgtgggactgagtggcaagtccctttgtgacttcaagaaccctgacttctctttgtgcagagaccagcccacccctgtgcccaccatgaccctcttcctcatgctgaactgcattccttccccaatcacctttcctgttccagaaaaggggctgggatgtctccgtctctgtctcaaatttgtggtccactgagctataacttacttctgtattaaaattagaatctgagtataaatttactttttcaaattatttccaagagagattgatgggttaattaaaggagaagattcctgaaatttgagagacaaaataaatggaagacatgagaacttt

SEQ ID NO: 10 - DNA, 948 bp, homo sapiens
cacacaccttaaccctgactttttttgctccagtttttcagaagaagtgaagtcaagatgaagaaccatttgcttttctggggagtcctggcggtttttattaaggctgttcatgtgaaagcccaagaagatgaaaggattgttcttgttgacaacaaatgtaagtgtgcccggattacttccaggatcatccgttcttccgaagatcctaatgaggacattgtggagagaaacatccgaattattgttcctctgaacaacagggagaatatctctgatcccacctcaccattgagaaccagatttgtgtaccatttgtctgacctctgtaaaaaatgtgatcctacagaagtggagctggataatcagatagttactgctacccagagcaatatctgtgatgaagacagtgctacagagacctgctacacttatgacagaaacaagtgctacacagctgtggtcccactcgtatatggtggtgagaccaaaatggtggaaacagccttaaccccagatgcctgctatcctgactaatttaagtcattgctgactgcatagctctttttcttgagaggctctccattttgattcagaaagttagcatatttattaccaatgaatttgaaaccagggctttttttttttttttgggtgatgtaaaaccaactccctgccaccaaaataattaaaatagtcacattgttatctttattaggtaatcacttcttaattatatgttcatactctaagtatcaaaatcttccaattatcatgctcacctgaaagaggtatgctctcttaggaatacagtttctagcattaaacaaataaacaaggggagaaaataaaactcaaggagtgaaaatcaggaggtgtaataaaatgttcctcgcattcaaaaaaaaaaaaaaaaaaaaaaaaaaaaaaaaaaaaaaaaaaaaaaaa

SEQ ID NO: 11 - DNA, 947 bp, homo sapiens
ccatccctgagatctttttataaaaaacccagtctttgctgaccagacaaagcataccagatctcaccagagagtcctaggggactacagaaggaaaaagacaagaggcagtaggatatctgtgtgtcctcccgctgaccacacttcctttagtgacccgattgcctcctcaagtcgcagacactatgctgcctcccatggccctgcccagtgtgtcctggatgctgctttcctgcctcattctcctgtgtcaggttcaaggtgaagaaacccagaaggaactgccctctccacggatcagctgtcccaaaggctccaaggcctatggctccccctgctatgccttgtttttgtcaccaaaatcctggatggatgcagatctggcttgccagaagcggccctctggaaaactggtgtctgtgctcagtggggctgagggatccttcgtgtcctccctggtgaggagcattagtaacagctattcatacatctggattgggctccatgaccccacacagggctctgagcctgatggagatggatgggagtggagtagcactgatgtgatgaattactttgcatgggagaaaaatccctccaccatcttaaaccctggccactgtgggagcctgtcaagaagcacaggatttctgaagtggaaagattataactgtgatgcaaagttaccctatgtctgcaagttcaaggactagggcaggtgggaagtcagcagcctgagcttggcgtgcagctcatcatggacatgagaccagtgtgaagactcaccctggaagagaatattctccccaaactgccctacctgactaccttgtcatgatcctccttctttttcctttttcttcaccttcatttcaggcttttctctgtcttccatgtcttgagatctcagagaataataataaaaatgttactttatacgtaaaaaa

SEQ ID NO: 12 - DNA, 847 bp, homo sapiens
ccatccctgagatctttttataaaaaacccagtctttgctgaccagacaaagcataccagatctcaccagagagtcgcagacactatgctgcctcccatggccctgcccagtgtgtcctggatgctgctttcctgcctcattctcctgtgtcaggttcaaggtgaagaaacccagaaggaactgccctctccacggatcagctgtcccaaaggctccaaggcctatggctccccctgctatgccttgtttttgtcaccaaaatcctggatggatgcagatctggcttgccagaagcggccctctggaaaactggtgtctgtgctcagtggggctgagggatccttcgtgtcctccctggtgaggagcattagtaacagctattcatacatctggattgggctccatgaccccacacagggctctgagcctgatggagatggatgggagtggagtagcactgatgtgatgaattactttgcatgggagaaaaatccctccaccatcttaaaccctggccactgtgggagcctgtcaagaagcacaggatttctgaagtggaaagattataactgtgatgcaaagttaccctatgtctgcaagttcaaggactagggcaggtgggaagtcagcagcctgagcttggcgtgcagctcatcatggacatgagaccagtgtgaagactcaccctggaagagaatattctccccaaactgccctacctgactaccttgtcatgatcctccttctttttcctttttcttcaccttcatttcaggcttttctctgtcttccatgtcttgagatctcagagaataataataaaaatgttactttatacttaaaaaa

SEQ ID NO: 13 - DNA, 1069 bp, homo sapiens
gcccctggaggaactgaacccactatcggtcatggggccgagactaaatgtggcgggttgtctttaatctgctgccaagaggaaactcattcaggcaagttcagccctttatgaggaattcccctgtggtcacattccaattcctggacctgctgccaccctcagaactgcatgctccttcttcagactttctaagaatgactcaggtcattggtggagtgaagtcaagatttccaactcagtcacctgaagagatggagataccattcatggagctggaggtccctggagatttgggaattcagataacaagctaagataaggagtttgcctacctctgtcctagagcgaagcctgagccttgggcgcgcagcacaccacaagtatctgttactgtgttttgcagaagcttcaggcggggatataagccccacaaggaaagcgctgagcagaggaggcctcagcttgacctgcggcagtgcagcccttgggacttccctcgccttccacctcctgctcgtctgcttcacaagctatcgctatggtgttcgtgcgcaggccgtggcccgccttgaccacagtgcttctggccctgctcgtctgcctaggggcgctggtcgacgcctaccccatcaaacccgaggctccccgcgaagacgcctcgccggaggagctgaaccgctactacgcctccctgcgccactacctcaacctggtcacccggcagcggtatgggaaaagagacggcccggacacgcttctttccaaaacgttcttccccgacggcgaggaccgccccgtcaggtcgcggtcggagggcccagacctgtggtgaggacccctgaggcctcctgggagatctgccaaccacgcccacgtcatttgcatacgcactcccgaccccagaaacccggattctgcctcccgacggcggcgtctgggcagggttcgggtgcggccctccgcccgcgtctcggtgcccccgccccctgggctggagggctgtgtgtggtccttccctggtcccaaaataaagagcaaattccacagaaaaaaaaaaaaaaaaaaa

SEQ ID NO: 14 - DNA, 808 bp, homo sapiens
gatataaagctcctacagctacctggcctgagaagccaactcagactcagccaacagagattgttgatttgcctcttaagcaagagattcattgcagctcagcatggctcagaccagctcatacttcatgctgatctcctgcctgatgtttctgtctcagagccaaggccaagaggcccagacagagttgccccaggcccggatcagctgcccagaaggcaccaatgcctatcgctcctactgctactactttaatgaagaccgtgagacctgggttgatgcagatctctattgccagaacatgaattcgggcaacctggtgtctgtgctcacccaggccgagggtgcctttgtggcctcactgattaaggagagtggcactgatgacttcaatgtctggattggcctccatgaccccaaaaagaaccgccgctggcactggagcagtgggtccctggtctcctacaagtcctggggcattggagccccaagcagtgttaatcctggctactgtgtgagcctgacctcaagcacaggattccagaaatggaaggatgtgccttgtgaagacaagttctcctttgtctgcaagttcaaaaactagaggcaactggaaaatacatgtctagaactgatccagcaattacaacggagtcaaaaattaaaccggaccatctctccaactcaactcaacctggacactctcttctctgctgagtttgccttgttaatcttcaatagttttacctaccccagtctttggaaccctaaataataaaaataaacatgtttccactattgtgctgtc

SEQ ID NO: 15 - DNA, 807 bp, homo sapiens
aaaccataccatatcccaccagagagtgactcctgattgcctcctcaagtcgcagacactatgctgcctcccatggccctgcccagtgtatcttggatgctgctttcctgcctcatgctgctgtctcaggttcaaggtgaagaaccccagagggaactgccctctgcacggatccgctgtcccaaaggctccaaggcctatggctcccactgctatgccttgtttttgtcaccaaaatcctggacagatgcagatctggcctgccagaagcggccctctggaaacctggtgtctgtgctcagtggggctgagggatccttcgtgtcctccctggtgaagagcattggtaacagctactcatacgtctggattgggctccatgaccccacacagggcaccgagcccaatggagaaggttgggagtggagtagcagtgatgtgatgaattactttgcatgggagagaaatccctccaccatctcaagccccggccactgtgcgagcctgtcgagaagcacagcatttctgaggtggaaagattataactgtaatgtgaggttaccctatgtctgcaagttcactgactagtgcaggagggaagtcagcagcctgtgtttggtgtgcaactcatcatgggcatgagaccagtgtgaggactcaccctggaagagaatattcgcttaattcccccaacctgaccacctcattcttatctttcttctgtttcttcctccccgctgtcatttcagtctcttcattttgtcatacggcctaaggctttaaagagcaataaaatttttagtctgc

SEQ ID NO: 16 - DNA, 784 bp, homo sapiens
aaaccataccatatcccaccagagagtcgcagacactatgctgcctcccatggccctgcccagtgtatcttggatgctgctttcctgcctcatgctgctgtctcaggttcaaggtgaagaaccccagagggaactgccctctgcacggatccgctgtcccaaaggctccaaggcctatggctcccactgctatgccttgtttttgtcaccaaaatcctggacagatgcagatctggcctgccagaagcggccctctggaaacctggtgtctgtgctcagtggggctgagggatccttcgtgtcctccctggtgaagagcattggtaacagctactcatacgtctggattgggctccatgaccccacacagggcaccgagcccaatggagaaggttgggagtggagtagcagtgatgtgatgaattactttgcatgggagagaaatccctccaccatctcaagccccggccactgtgcgagcctgtcgagaagcacagcatttctgaggtggaaagattataactgtaatgtgaggttaccctatgtctgcaagttcactgactagtgcaggagggaagtcagcagcctgtgtttggtgtgcaactcatcatgggcatgagaccagtgtgaggactcaccctggaagagaatattcgcttaattcccccaacctgaccacctcattcttatctttcttctgtttcttcctccccgctgtcatttcagtctcttcattttgtcatacggcctaaggctttaaagagcaataaaatttttagtctgc

SEQ ID NO: 17 - DNA, 1002 bp, homo sapiens
gggagggtcccttcctcagggagcacaggaactctgagactcagcaagggtgtcctgggagggctcggggatgggagagtacacagattcacaactcattcagaactgtagaagatgatggatgtgaccaagatcactttagtcctaggggactagagaaggaaaatgacatgaggcagtggggtatctgtgtgttctcccactgaccacgctttctttagtgactcctgattgcctcctcaagtcgcagacactatgctgcctcccatggccctgcccagtgtatcttggatgctgctttcctgcctcatgctgctgtctcaggttcaaggtgaagaaccccagagggaactgccctctgcacggatccgctgtcccaaaggctccaaggcctatggctcccactgctatgccttgtttttgtcaccaaaatcctggacagatgcagatctggcctgccagaagcggccctctggaaacctggtgtctgtgctcagtggggctgagggatccttcgtgtcctccctggtgaagagcattggtaacagctactcatacgtctggattgggctccatgaccccacacagggcaccgagcccaatggagaaggttgggagtggagtagcagtgatgtgatgaattactttgcatgggagagaaatccctccaccatctcaagccccggccactgtgcgagcctgtcgagaagcacagcatttctgaggtggaaagattataactgtaatgtgaggttaccctatgtctgcaagttcactgactagtgcaggagggaagtcagcagcctgtgtttggtgtgcaactcatcatgggcatgagaccagtgtgaggactcaccctggaagagaatattcgcttaattcccccaacctgaccacctcattcttatctttcttctgtttcttcctccccgctgtcatttcagtctcttcattttgtcatacggcctaaggctttaaagagcaataaaatttttagtctgc

SEQ ID NO: 18 - DNA, 1285 bp, homo sapiens
ataagacttttatggatggattgtttttctcaaataatattatcgctttgtgactaaagtaaagattattaattcctgaggcaagaagatataaaagctccagaaacgttgactgggaccactggagacactgaagaaggcaggggcccttagagtcttggttgccaaacagatttgcagatcaaggagaacccaggagtttcaaagaagcgctagtaaggtctctgagatccttgcactagctacatcctcagggtaggaggaagatggcttccagaagcatgcggctgctcctattgctgagctgcctggccaaaacaggagtcctgggtgatatcatcatgagacccagctgtgctcctggatggttttaccacaagtccaattgctatggttacttcaggaagctgaggaactggtctgatgccgagctcgagtgtcagtcttacggaaacggagcccacctggcatctatcctgagtttaaaggaagccagcaccatagcagagtacataagtggctatcagagaagccagccgatatggattggcctgcacgacccacagaagaggcagcagtggcagtggattgatggggccatgtatctgtacagatcctggtctggcaagtccatgggtgggaacaagcactgtgctgagatgagctccaataacaactttttaacttggagcagcaacgaatgcaacaagcgccaacacttcctgtgcaagtaccgaccatagagcaagaatcaagattctgctaactcctgcacagccccgtcctcttcctttctgctagcctggctaaatctgctcattatttcagaggggaaacctagcaaactaagagtgataagggccctactacactggcttttttaggcttagagacagaaactttagcattggcccagtagtggcttctagctctaaatgtttgccccgccatccctttccacagtatccttcttccctcctcccctgtctctggctgtctcgagcagtctagaagagtgcatctccagcctatgaaacagctgggtctttggccataagaagtaaagatttgaagacagaaggaagaaactcaggagtaagcttctagaccccttcagcttctacacccttctgccctctctccattgcctgcaccccaccccagccactcaactcctgcttgtttttcctttggccataggaaggtttaccagtagaatccttgctaggttgatgtgggccatacattcctttaataaaccattgtgtacataagaaaaaaaaaa

SEQ ID NO: 19 - protein, 224 aa, homo sapiens
MRVSGVLRLLALIFAIVTTWMFIRSYMSFSMKTIRLPRWLAASPTKEIQVKKYKCGLIKPCPANYFAFKICSGAANVVGPTMCFEDRMIMSPVKNNVGRGLNIALVNGTTGAVLGQKAFDMYSGDVMHLVKFLKEIPGGALVLVASYDDPGTKMNDESRKLFSDLGSSYAKQLGFRDSWVFIGAKDLRGKSPFEQFLKNSPDTNKYEGWPELLEMEGCMPPKPF

SEQ ID NO: 20 - protein, 396 aa, homo sapiens
MFLKAVVLTLALVAVAGARAEVSADQVATVMWDYFSQLSNNAKEAVEHLQKSELTQQLNALFQDKLGEVNTYAGDLQKKLVPFATELHERLAKDSEKLKEEIGKELEELRARLLPHANEVSQKIGDNLRELQQRLEPYADQLRTQVSTQAEQLRRQLTPYAQRMERVLRENADSLQASLRPHADELKAKIDQNVEELKGRLTPYADEFKVKIDQTVEELRRSLAPYAQDTQEKLNHQLEGLTFQMKKNAEELKARISASAEELRQRLAPLAEDVRGNLRGNTEGLQKSLAELGGHLDQQVEEFRRRVEPYGENFNKALVQQMEQLRQKLGPHAGDVEGHLSFLEKDLRDKVNSFFSTFKEKESQDKTLSLPELEQQQEQQQEQQQEQVQMLAPLES

SEQ ID NO: 21 - protein, 101 aa, homo sapiens
MGTRLLPALFLVLLVLGFEVQGTQQPQQDEMPSPTFLTQVKESLSSYWESAKTAAQNLYEKTYLPAVDEKLRDLYSKSTAAMSTYTGIFTDQVLSVLKGEE

SEQ ID NO: 22 - protein, 115 aa, homo sapiens
MNSGVCLCVLMAVLAAGALTQPVPPADPAGSGLQRAEEAPRRQLRVSQRTDGESRAHLGALLARYIQQARKAPSGRMSIVKNLQNLDPSHRISDRDYMGWMDFGRRSAEEYEYPS

SEQ ID NO: 23 - protein, 301 aa, homo sapiens
MLPLTMTVLILLLLPTGQAAPKDGVTRPDSEVQHQLLPNPFQPGQEQLGLLQSYLKGLGRTEVQLEHLSREQVLLYLFALHDYDQSGQLDGLELLSMLTAALAPGAANSPTTNPVILIVDKVLETQDLNGDGLMTPAELINFPGVALRHVEPGEPLAPSPQEPQAVGRQSLLAKSPLRQETQEAPGPREEAKGQVEARRESLDPVQEPGGQAEADGDVPGPRGEAEGQAEAKGDAPGPRGEAGGQAEAEGDAPGPRGEAGGQAEARENGEEAKELPGETLESKNTQNDFEVHIVQVENDEI

SEQ ID NO: 24 - protein, 216 aa, homo sapiens
MRSGCVVVHVWILAGLWLAVAGRPLAFSDAGPHVHYGWGDPIRLRHLYTSGPHGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 25 - protein, 115 aa, homo sapiens
MNAFLLSALCLLGAWAALAGGVTVQDGNFSFSLESVKKLKDLQEPQEPRVGKLRNFAPIPGEPVVPILCSNPNFPEELKPLCKEPNAQEILQRLEEIAEDPGTCEICAYAACTGC

SEQ ID NO: 26 - protein, 262 aa, homo sapiens
MRNSYRFLASSLSVVVSLLLIPEDVCEKIIGGNEVTPHSRPYMVLLSLDRKTICAGALIAKDWVLTAAHCNLNKRSQVILGAHSITREEPTKQIMLVKKEFPYPCYDPATREGDLKLLQLTEKAKINKYVTILHLPKKGDDVKPGTMCQVAGWGRTHNSASWSDTLREVNITIIDRKVCNDRNHYNFNPVIGMNMVCAGSLRGGRDSCNGDSGSPLLCEGVFRGVTSFGLENKCGDPRGPGVYILLSKKHLNWIIMTIKGAV

SEQ ID NO: 27 - protein, 338 aa, homo sapiens
MVVMAPRTLFLLLSGALTLTETWAGSHSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWKQSSLPTIPIMGIVAGLVVLAAVVTGAAVAAVLWRKKSSD

SEQ ID NO: 28 - protein, 159 aa, homo sapiens
MKNHLLFWGVLAVFIKAVHVKAQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPD

SEQ ID NO: 29 - protein, 175 aa, homo sapiens
MLPPMALPSVSWMLLSCLILLCQVQGEETQKELPSPRISCPKGSKAYGSPCYALFLSPKSWMDADLACQKRPSGKLVSVLSGAEGSFVSSLVRSISNSYSYIWIGLHDPTQGSEPDGDGWEWSSTDVMNYFAWEKNPSTILNPGHCGSLSRSTGFLKWKDYNCDAKLPYVCKFKD

SEQ ID NO: 30 - protein, 97 aa, homo sapiens
MVFVRRPWPALTTVLLALLVCLGALVDAYPIKPEAPREDASPEELNRYYASLRHYLNLVTRQRYGKRDGPDTLLSKTFFPDGEDRPVRSRSEGPDLW

SEQ ID NO: 31 - protein, 166 aa, homo sapiens
MAQTSSYFMLISCLMFLSQSQGQEAQTELPQARISCPEGTNAYRSYCYYFNEDRETWVDADLYCQNMNSGNLVSVLTQAEGAFVASLIKESGTDDFNVWIGLHDPKKNRRWHWSSGSLVSYKSWGIGAPSSVNPGYCVSLTSSTGFQKWKDVPCEDKFSFVCKFKN

SEQ ID NO: 32 - protein, 175 aa, homo sapiens
MLPPMALPSVSWMLLSCLMLLSQVQGEEPQRELPSARIRCPKGSKAYGSHCYALFLSPKSWTDADLACQKRPSGNLVSVLSGAEGSFVSSLVKSIGNSYSYVWIGLHDPTQGTEPNGEGWEWSSSDVMNYFAWERNPSTISSPGHCASLSRSTAFLRWKDYNCNVRLPYVCKFTD

SEQ ID NO: 33 - protein, 158 aa, homo sapiens
MASRSMRLLLLLSCLAKTGVLGDIIMRPSCAPGWFYHKSNCYGYFRKLRNWSDAELECQSYGNGAHLASILSLKEASTIAEYISGYQRSQPIWIGLHDPQKRQQWQWIDGAMYLYRSWSGKSMGGNKHCAEMSSNNNFLTWSSNECNKRQHFLCKYRP

SEQ ID NO: 34 - DNA, 20 bp, artificial
  primer
ctgcccagccaactactttg

SEQ ID NO: 35 - DNA, 19 bp, artificial
  primer
ctcccgtggttccattcac

SEQ ID NO: 36 - DNA, 21 bp, artificial
  primer
ccaagatcgaccagaacgtgg

SEQ ID NO: 37 - DNA, 20 bp, artificial
  primer
gtcctgagcatagggagcca

SEQ ID NO: 38 - DNA, 20 bp, artificial
  primer
cccagagcaaaaagcgactc

SEQ ID NO: 39 - DNA, 24 bp, artificial
  primer
accatccatgcctacaacaaaagg

SEQ ID NO: 40 - DNA, 21 bp, artificial
  primer
caacaggctgaaggctacgat

SEQ ID NO: 41 - DNA, 21 bp, artificial
  primer
cgatttttgcggagaccttgg

SEQ ID NO: 42 - DNA, 21 bp, artificial
  primer
cagaagtgcgaagaggaggtc

SEQ ID NO: 43 - DNA, 22 bp, artificial
  primer
tcatcttagaggccacgaacat

SEQ ID NO: 44 - DNA, 18 bp, artificial
  primer
tccagccaatgcctttgc

SEQ ID NO: 45 - DNA, 24 bp, artificial
  primer
tggagattacttttcagtgcagaa

SEQ ID NO: 46 - DNA, 22 bp, artificial
  primer
tagagatcgacgggatgaaact

SEQ ID NO: 47 - DNA, 19 bp, artificial
  primer
cagatccgccactccttct

SEQ ID NO: 48 - DNA, 21 bp, artificial
  primer
tggtggatgggaagctgtcta

SEQ ID NO: 49 - DNA, 21 bp, artificial
  primer
ttcttgcggtaggctgcatag

SEQ ID NO: 50 - DNA, 22 bp, artificial
  primer
gactcttgcgtcaacttcaagg

SEQ ID NO: 51 - DNA, 21 bp, artificial
  primer
caggctgtcttttgtcaacga

SEQ ID NO: 52 - DNA, 22 bp, artificial
  primer
atacaagctcacgtactccact

SEQ ID NO: 53 - DNA, 21 bp, artificial
  primer
tccacagtaacacaacgtcca

SEQ ID NO: 54 - DNA, 22 bp, artificial
  primer
tttggagtggtagtaaaaaggg

SEQ ID NO: 55 - DNA, 22 bp, artificial
  primer
tgacatcagggactcagagtag

SEQ ID NO: 56 - DNA, 19 bp, artificial
  primer
ccggagacccttagatcga

SEQ ID NO: 57 - DNA, 26 bp, artificial
  primer
tagcctgtaaaagatttctgcaaacc

SEQ ID NO: 58 - DNA, 23 bp, artificial
  primer
ccgaaacccatcagtccgtttaa

SEQ ID NO: 59 - DNA, 22 bp, artificial
  primer
acataggtggctgtccagacaa

(19) United States
(12) Patent Application Publication
(10) Pub. No.: US 2011/0107439 A1
(43) Pub. Date: May 5, 2011

(54) DIAGNOSTIC OF PRE-SYMPTOMATIC METABOLIC SYNDROME

(75) Inventors: Nicole Johanna Wilhelmina De Wit, Vorstenbosch (NL); Roelof Van Der Meer, Ede (NL)

(73) Assignee: PODICEPS B.V., Houten (NL)

(21) Appl. No.: 12/933,762

(22) PCT Filed: Mar. 17, 2009
(86) PCT No.: PCT/NL2009/050126

§ 371 Date: Jan. 19, 2011

Related U.S. Application Data
(60) Provisional application No. 61/038,433, filed on Mar. 21, 2008.

(30) Foreign Application Priority Data
May 15, 2008 (EP) 08156294.4

(51) Int. Cl. C12Q 1/68; C07K 16/00; G01N 33/53; A61K 49/00

(52) U.S. Cl. 800/3; 435/6; 530/387.9; 436/501

(57) ABSTRACT

The invention relates to a method for diagnosing pre-symptomatic metabolic syndrome in a subject, wherein said method comprises determining the expression level of a gene represented by a sequence selected from the group consisting of SEQ ID NO: 1-18 in a subject. The invention described target genes for preventing or stopping further progress of metabolic syndrome into clinical disease states.

### FIELD OF THE INVENTION

The invention relates to a method for diagnosing pre-symptomatic metabolic syndrome in a subject, wherein said method comprises determining the expression level of a gene represented by a nucleotide sequence selected from the group consisting of SEQ ID NO:1-18 in a subject.

### BACKGROUND OF THE INVENTION

Metabolic syndrome is a multi-component condition associated with a high risk of type 2 diabetes mellitus and cardiovascular disease (38) and the onset of cancer. In the industrialized societies, approximately 20-40% of the population are affected by the metabolic syndrome and its incidence is expected to rise even further in the next decades (31). Obesity and insulin resistance are two major risk factors underlying the metabolic syndrome. Obesity is considered the principal instigator that predisposes to insulin resistance, which is the pivotal metabolic disturbance in the metabolic syndrome (25).

Lifestyle factors, such as nutrition and limited physical activity, are known to contribute to the pathogenesis of obesity and insulin resistance. The association between development of these disorders and excessive intake of dietary fat is frequently studied, especially in obesity-prone C57BL/6J mice (2, 27, 34, 43, 51). Most of these studies are focused on the physiology and underlying molecular mechanisms in liver, skeletal muscle and adipose tissue, as these organs are target organs of insulin-modulated metabolism (6, 30, 46). However, there is growing evidence that also the small intestine can play an essential role in the etiology of obesity and/or insulin resistance, due to its gatekeeper function at the physical interphase between body and diet. Next to an efficient uptake of nutrients, the enterocytes in the small intestine are also responsible for sensing of luminal contents that are modulated by the diet. As a result of this sensing, the small intestine secretes signaling molecules, such as gut hormones and pro- and anti-inflammatory cytokines, to which liver, muscle and adipose tissue can respond by modulating their metabolism to keep homeostatic control. Potential small intestinal factors that contribute to development of metabolic syndrome are specific effects of gut hormones on satiety and glucose homeostasis (9, 12), diminished fatty acid oxidative capacity of enterocytes (27) and gut microbiota composition (2, 48).

Due to the growing importance of metabolic syndrome in western societies, there is a great need for specific markers that could be used in a method for diagnosing pre-symptomatic metabolic syndrome in a subject. Such markers are not available yet.

### DESCRIPTION OF THE INVENTION

In this inventory study, we investigated the potential role of the small intestine in development of dietary fat-induced obesity and/or insulin resistance in C57BL/6J mice in a rather comprehensive way during time. Therefore, we performed microarray analysis of small intestinal mucosa to explore fat-modulated biological processes and an additional ‘secretome’ analysis to identify secreted proteins that are able to induce systemic effects underlying the etiology of the metabolic syndrome. Surprisingly, we found that 15 genes among other a Fam3D and/or a ApoA4 gene could be used as specific markers in a method for diagnosing pre-symptomatic metabolic syndrome in a subject.

### Diagnostic Method

In a first aspect, there is provided a method for diagnosing pre-symptomatic metabolic syndrome in a subject, the method comprising the steps of:
(a) determining the expression level of a gene represented by a nucleotide sequence selected from the group consisting of SEQ ID NO:1-18 in a subject; and,
(b) comparing the expression level of said gene as defined in (a) with a reference value for said expression level, the reference value preferably being the average value for said expression level in a control subject.

In the context of the invention, metabolic syndrome may be defined as being a multi-component condition associated with a high risk of type 2 diabetes mellitus and cardiovascular disease. Symptomatic metabolic syndrome is generally characterized by at least one of obesity, insulin resistance, type 2 diabetes mellitus and a (cardio)vascular disease. Several methods are already known to diagnose metabolic syndrome (Grundy et al (2004) Circulation, 109: 433-438 and Grundy et al (2005) Circulation, 112: 2735-2752). Each of these methods define a combination of parameters, for which a specific value or range for each of the parameters will establish the diagnosis of metabolic syndrome in a subject. For example the National Cholesterol Education Program's Adult Treatment Panel III report (so-called ATP III) defines that when at least three of the following parameters being body weight, lipid concentration, blood pressure, glucose are comprised within a specific range as defined in table 1 of Grundy et al (2004) or in table 1 of Grundy et al (2005), metabolic syndrome will be diagnosed. As another example, the World Health Organization (WHO) proposed another definition wherein the presence of insulin resistance, in combination with two of the following parameters being body weight, lipid, blood pressure and glucose being comprised within a specific range as defined in table 2 of Grundy et al (2004) or table 1 of Grundy et al (2005) metabolic syndrome is diagnosed. Using any of the existing methods (such as ATP III or WHO definitions) for diagnosing metabolic syndrome leads to a relative late diagnosis of the syndrome, which means that the course of the syndrome is quite difficult to be reversed in a subject.

In the context of the invention, diagnosing pre-symptomatic metabolic syndrome preferably means that a diagnosis is reached before the actual development of a symptomatic metabolic syndrome as earlier defined herein. The invention allows a specific and early detection of metabolic syndrome, which will allow to reverse the course of the syndrome more easily in a subject. In addition, the target genes or proteins identified in the invention may be effected by other means to reverse or stop the development of metabolic syndrome and the related diseases. The invention is the first known to allow a detection of a pre-symptomatic metabolic syndrome. A detection of a pre-symptomatic metabolic syndrome is preferably reached earlier in time than the detection of symptomatic metabolic syndrome using any of the other methods (or definitions) earlier defined herein. In this context, “earlier in time” preferably means at least one day, at least two days, at least three days, at least four days, at least five days, at least six days, at least seven days, at least eight days, at least nine days, at least ten days at least 15 days, at least 20 days, at least 25 days, at least 30 days, at least 1 month, at least 2 months, at least 3 months, at least 4 months, at least 5 months, at least 6 months, at least 7 months, at least 8 months, at least 9 months, at least 10 months, at least 11 months, at least 12 months or more before the actual development of a symptomatic metabolic syndrome.

In the context of the invention, diagnosis preferably means a predictive risk assessment of the subsequent development of metabolic syndrome in a subject.

In the context of the invention, a subject may be an animal or a human being. In principle, any subject could be diagnosed using the method of the invention. The diagnosis method may be applied as often as necessary in a subject. Preferably, a subject diagnosed is a subject suspected to have a high risk of developing a metabolic syndrome, due for example to potential genetic predisposition, and/or to the age of the subject and/or to the lifestyle of a subject (for example nutritional habit and/or to the absence of physical activity). Preferably, a subject is a human being.

In the context of the invention, “a gene or nucleotide molecule as identified herein” preferably means a gene or nucleotide molecule represented by a nucleotide sequence selected from the group consisting of SEQ ID NO:1-18, more preferably from the group consisting of SEQ ID NO:1, 2, 3, 4, 5, 6, 7, 8, 9, 10, 11 or 12, 13, 14, 15 or 16 or 17 and 18. Even more preferably, the group consists of SEQ ID NO:1 and 2.

In the context of the invention, “a polypeptide or protein as identified herein” preferably means a polypeptide encoded by a gene or nucleotide molecule as identified herein.

In the context of the invention, “a reference value” for the expression level of a gene as identified herein is preferably the average value for said expression level in control subjects. More preferably, a control subject is a subject, who has not developed a metabolic syndrome as diagnosed by any of the methods as mentioned earlier herein. Alternatively according to an even more preferred embodiment, a control subject is a subject who has not developed any of the characteristics (i.e. parameters) of the metabolic syndrome yet. For example, a subject will not be said to have abdominal obesity, lipid, blood pressure, glucose and/or insulin resistance as defined in table 1 of Grundy et al (2005).

The assessment of the expression level of a gene as identified herein may be realised at the protein expression level (quantifying the amount of a protein encoded by said genes as identified herein), and/or by quantifying the amount of a gene (or nucleotide molecule) encoding said protein (both the reference value from a control subject and the value from a subject wherein the method is being carried out). Table 5 (and genes marked in grey in table 4) identifies 15 genes represented by 18 nucleotide sequences SEQ ID NO:1-18 and corresponding encoded polypeptides or proteins. Each of these genes can be used alone or in combination or in sub-combinations as a marker for pre-symptomatic metabolic syndrome. They were all found up-regulated in the studied animal model, their expression product is secreted and detectable in blood and their expression is restricted to a limited number of tissues. Each of these features renders these genes attractive to be used as a marker for diagnosing pre-symptomatic metabolic syndrome and as target for interfering in the development of full blown metabolic syndrome and consequentially the related diseases. The invention encompasses the use of 1, 2, 3, 4, 5, 6, 7, 8, 9, 10, 11, 12, 13, 14 till 15 genes represented by SEQ ID NO:1-18. It is to be noted that both genes are represented by more than one nucleotide sequences: the Pap gene is represented by two nucleotides sequences SEQ ID NO:11 and 12, the Reg3g gene by three SEQ ID NO:15, 16 and 17. These two (respectively three) nucleotide sequences code for one polypeptide represented by the same amino acid sequence SEQ ID NO:29 (respectively SEQ ID NO:32). Therefore, when referring to the Pap (respectively the Reg3g) gene, one may use either of the identified nucleotide sequences. Fam3D (Oit1, represented by SEQ ID NO:1) and ApoA4 (represented by SEQ ID NO:2) are gut-specific markers (small intestine), their differences in gene expression as measured in serum may easily be extrapolated to differences in gene expression in the small intestine. Therefore, the use of these genes represented by SEQ ID NO:1 and/or SEQ ID NO:2 is preferred in a diagnostic method for pre-symptomatic metabolic syndrome.

The skilled person will understand that for each identified gene (or nucleotide sequence) and corresponding polypeptide or protein, it is possible to isolate multiple isoforms of a given protein depending on the subject to be tested. It is to be understood that each gene as identified herein by a given Sequence Identity Number (SEQ ID NO) is not limited to this specific sequence. Each gene sequence or nucleotide sequence as identified herein encodes a given protein or polypeptide as identified in table 5. Throughout this application, each time one refers to a specific nucleotide sequence SEQ ID NO (take SEQ ID NO:1 as example), one may replace it by:

i. a polypeptide comprising an amino acid sequence that has at least 60% sequence identity with amino acid sequence SEQ ID NO:19 (as identified in table 5) as being encoded by SEQ ID NO:1,

ii. a nucleotide sequence comprising a nucleotide sequence that has at least 60% sequence identity with SEQ ID NO:1 (as example).

iii. a nucleotide sequence the complementary strand of which hybridizes to a nucleotide sequence of (ii);

iv. a nucleotide sequence the sequence of which differs from the sequence of a nucleotide sequence of (iii) due to the degeneracy of the genetic code.

iv. a nucleotide sequence that encodes an amino acid sequence that has at least 60% amino acid identity with an amino acid sequence encoded by a nucleotide sequence SEQ ID NO:1.

Each nucleotide sequence or amino acid sequence described herein by virtue of its identity percentage (at least 60%) with a given nucleotide sequence or amino acid sequence respectively has in a further preferred embodiment an identity of at least 65%, 70%, 75%, 80%, 85%, 90%, 95%, 97%, 98%, 99% or more identity with the given nucleotide or amino acid sequence respectively. In a preferred embodiment, sequence identity is determined by comparing the whole length of the sequences as identified herein.
Identity is later herein defined. The quantification of the amount of a gene (or nucleotide molecule) as identified herein is preferably performed using classical molecular biology techniques such as (real time) PCR, arrays or northern analysis. In this embodiment, a gene (or nucleotide molecule) encoding a polypeptide as defined herein means a messenger RNA (mRNA). Alternatively, according to another preferred embodiment, in the diagnosis method the expression level of a polypeptide is determined directly by quantifying the amount of said polypeptide. Quantifying a polypeptide amount may be carried out by any known technique. Preferably, a polypeptide amount is quantified using a molecule which specifically binds to said polypeptide. Preferred binding molecules are selected from: an antibody, which has been specifically raised for recognizing a given polypeptide, any other molecule which is known to specifically bind said polypeptide. Such antibody could be used in any immunoassay known to the skilled person such as western blotting, or ELISA (Enzyme-Linked Immuno Sorbent Assay) or FACS (Fluorescence Activated Cell Sorting) using latex beads. The preparation of an antibody is known to those skilled in the art. A short explanation of methods that could be used to prepare antibodies is later herein given. An example of a suitable specific antibody raised against Fam3D is described in US2005/0158753. In the context of the invention, any other molecule known to bind a given polypeptide may be a nucleic acid, e.g. a DNA regulatory region, a polypeptide, a metabolite, a substrate, a regulatory element, a structural component, a chaperone (transport) molecule, a peptide mimetic, a non-peptide mimetic, or any other type of ligand. Mimetic is later herein defined. Binding of a given polypeptide to a second binding molecule may be detected by any standard methods known to those skilled in the art. Suitable methods include affinity chromatography co-electrophoresis (ACE) assays and ELISA. The skilled person will understand that alternatively or in combination with the quantification of a gene encoding a given polypeptide and/or the corresponding polypeptide, the quantification of a substrate of the corresponding polypeptide or of any compound known to be associated with the function of the corresponding polypeptide or the quantification of the function or activity of the corresponding polypeptide using a specific assay is encompassed within the scope of the diagnosis method of the invention. For example, transactivation of a target gene by Fam3D or a Fam3D binding molecule can be determined and quantified, e.g., in a transient transfection assay in which the promoter of the target gene is linked to a reporter gene, e.g., P-galactosidase or luciferase.
Such evaluations can be done in vitro or in vivo or ex vivo.

Since the expression level of a gene (or nucleotide molecule) encoding a polypeptide as identified herein and/or amounts of corresponding polypeptide may be difficult to detect in a subject, a sample from a subject is preferably used. According to another preferred embodiment, the expression level (of a gene or nucleotide molecule or polypeptide) is determined ex vivo in a sample obtained from a subject. A sample preferably comprises or consists of a solid of a semi solid sample. Preferred solid or semi solid samples include a part of the small intestine of a subject, also called an intestinal biopsy. Alternatively, a sample preferably comprises or consists of a fluid obtained from a subject. More preferably, a fluid comprises or consists of or is selected from: urine, faeces, blood or saliva. Even more preferably, a fluid is blood plasma. Subsequently, a nucleotide molecule encoding a polypeptide as identified herein and/or said polypeptide are extracted and optionally purified using known methods to the skilled person.

In a more preferred diagnosis method, pre-symptomatic metabolic syndrome is diagnosed when the comparison leads to the finding of a detectable expression of a (i.e. at least one) gene (or nucleotide molecule) and/or of a corresponding polypeptide as identified herein. Alternatively or in combination with earlier preferred embodiment, the comparison leads to the finding of an increase of the expression level of a (i.e. at least one) gene (or nucleotide molecule) and/or of a corresponding polypeptide as identified herein. In control subjects as defined before, the expression of said gene (or nucleotide molecule) and/or corresponding polypeptide is preferably significantly lower than in subjects diagnosed as having a pre-symptomatic metabolic syndrome.

Detection or an increase of the expression level of a polypeptide as identified herein and/or an increase or a detection of the expression level of a gene (or nucleotide molecule) encoding said polypeptide (or steady state level of said polypeptide) is preferably defined as being a detectable change of the expression level of said polypeptide and/or of a nucleotide molecule encoding said polypeptide (or steady state level of the encoded polypeptide or any detectable change in the biological activity of a polypeptide as defined herein) using a method as defined earlier on as compared to the expression level of a polypeptide as identified herein and/or of a corresponding gene (or nucleotide molecule) (or steady state level of the corresponding encoded polypeptide) in a control subject. According to a preferred embodiment, detection or an increase of the expression level of a gene (or nucleotide molecule) as identified herein is quantified using a specific mRNA assay for the gene (or nucleotide molecule) as earlier defined herein. Preferably, an increase of the expression level of a gene (or nucleotide molecule) encoding a polypeptide as identified herein means an increase of at least 5% of the expression level of the gene (or nucleotide molecule) using PCR. For example, preferred primers used for the PCR for the detection of the expression of a Fam3D gene are identified as SEQ ID NO:34 5′-CTGCCCAGCCAACTACTTTG-3′ and SEQ ID NO:35 5′-CTCCCGTGGTTCCATTCAC-3′. More preferably, an increase of the expression level of a gene (or nucleotide molecule) means an increase of at least 10%, even more preferably at least 20%, at least 30%, at least 40%, at least 50%, at least 70%, at least 90%, at least 150% or more. Preferred primers for the PCR for the detection of the expression of a ApoA4 gene are identified as SEQ ID NO:36 5′-CCAAGATCGACCAGAACGTGG-3′ and SEQ ID NO:37 5′-GTCCTGAGCATAGGGAGCCA-3′.

Preferably, an increase of the expression level of a polypeptide as identified herein means an increase of at least 5% of the expression level of said polypeptide using western blotting and/or using ELISA or a suitable assay. More preferably, an increase of the expression level of a polypeptide means an increase of at least 10%, even more preferably at least 20%, at least 30%, at least 40%, at least 50%, at least 70%, at least 90%, at least 150% or more.

Preferably, an increase of an activity of a given polypeptide as identified herein means an increase of at least 5% of the polypeptide activity using a suitable assay. More preferably, an increase of a polypeptide activity means an increase of at least 10%, even more preferably at least 20%, at least 30%, at least 40%, at least 50%, at least 70%, at least 90%, at least 150% or more.

In a most preferred diagnosis method, pre-symptomatic metabolic syndrome is diagnosed when the comparison leads to the finding of a detectable expression of a Fam3D and/or ApoA4 using PCR and/or an increase of the expression level of a Fam3D and/or ApoA4, said detection or increase being detected at the level of a gene (or nucleotide molecule) encoding a Fam3D (respectively ApoA4) (mRNA) (an increase of at least 5% of the expression level of the gene or nucleotide molecule) using PCR primers as defined herein.

### Assay Device

In a second aspect, there is provided an assay device for diagnosing pre-symptomatic metabolic syndrome in a subject, wherein the device comprises a molecule which specifically binds a polypeptide as defined earlier herein. More preferably, a device comprises a molecule which specifically binds a Fam3D polypeptide and/or a molecule which specifically binds a ApoA4 polypeptide.

This device may be used in a diagnosis method of the invention. Any subject or physician could use this device at office/home, repeat the use of such device as often as necessary.

The type of molecules that are known to specifically bind a polypeptide as defined herein have already been earlier described herein. In a preferred embodiment, the molecule which specifically binds a polypeptide as identified herein and which is present in the device is an antibody.

In a preferred embodiment, an assay device is a lateral flow test strip also known as dipstick, preferably, though not necessarily, encased in a housing, designed to be read by the subject, and the assay is a sandwich immunoassay. Such devices are impregnated with reagents that specifically indicate the presence of a polypeptide as identified herein by changing colour upon contact with a sample. Preferred subject's samples have already been defined herein. The antibody is preferably labelled by conjugation to a physically detectable label, and upon contacting with the sample containing a polypeptide as identified herein forms a complex. The complex is then contacted with a second antibody, which recognizes the first antibody and which is immobilized on a solid support within the device. The second antibody captures the complex to form a sandwich complex, and the resulting sandwich complex, which is immobilized on the solid support, is detectable by virtue of the label. The test strip may then be inserted into a reader, where the signal from the label in the complex is measured. Alternatively, the test strip could be inserted into the reader prior to addition of the sample. Alternatively and according to a preferred embodiment, the presence of a polypeptide as identified herein is visualised by a subject as a change of colour of at least part of the device. Dipsticks are usually made of paper or cardboard. Usually additional molecules are present in said device as positive or negative controls. A typical positive control could be an antibody recognizing a molecule which is known to be present in a sample to be tested. A typical negative control could be an antibody recognizing a molecule which is known to be absent in a sample to be tested. Accordingly in a further aspect, there is provided the use of such assay device for diagnosing pre-symptomatic metabolic syndrome in a subject, wherein the device comprises a molecule which specifically binds a polypeptide as defined earlier herein. More preferably, a device comprises a molecule which specifically binds a Fam3D polypeptide and/or a molecule which specifically binds a ApoA4 polypeptide. A preferred molecule which specifically binds a Fam3D, respectively a ApoA4 polypeptide is an antibody, more preferably a monoclonal antibody. In another preferred embodiment, such assay is used in a method for diagnosing pre-symptomatic metabolic syndrome as identified herein.

### Method for Identification

In a further aspect, there is provided a method for identification of a substance capable of preventing, treating and/or delaying the progression of metabolic syndrome in a subject, the method comprising the steps of:
(a) providing a test cell population or a test animal capable of expressing a gene (or nucleotide molecule) encoding a polypeptide as identified herein and/or a gene (or nucleotide molecule) encoding said polypeptide;
(b) contacting the test cell population or the test animal with a substance;
(c) determining the expression level of a gene (or nucleotide molecule) encoding said polypeptide or the activity or steady state level of said polypeptide in a test cell population or in the test animal contacted with the substance;
(d) comparing the expression, activity or steady state level determined in (c) with the expression, activity or steady state level of the gene (or nucleotide molecule) or of the polypeptide in a test cell population or in a test animal that is not contacted with the substance; and,
(e) identifying a substance that produces a difference in expression level, activity or steady state level of the gene (or nucleotide molecule) or the polypeptide, between the test cell population or test animal that is contacted with the substance and the test cell population or test animal that is not contacted with the substance.

In a preferred embodiment, a test animal is a mouse, more preferably a C57BL/6J mouse. A preferred test cell population comprises mammalian cells, more preferably human cells. Even more preferred cells are colon carcinoma cell lines LS174T and LOVO, since they both express Fam3D.

Alternatively or in combination with previous preferred embodiment in a further preferred embodiment, in step (a), a test cell or a test animal has been modified to over-express a polypeptide as identified herein. This is preferably carried out by transforming a test cell with a nucleic acid construct comprising a nucleotide sequence encoding said polypeptide as defined herein. Alternatively, this is preferably carried out by generating a test animal being transgenic for a given polypeptide as identified herein and as later explained herein. In a further aspect, the invention relates to such a nucleic acid construct. Preferably, a nucleotide sequence is operably linked to a promoter that is capable of driving expression of a nucleotide sequence in a chosen test cell. In a preferred embodiment a nucleic acid construct is a viral gene therapy vector selected from gene therapy vectors based on an adenovirus, an adeno-associated virus (AAV), a herpes virus, a pox virus and a retrovirus. A preferred viral gene therapy vector is an AAV or Lentiviral vector. Such vectors are further described herein below.

Depending on the system used (test cell or test animal), the skilled person will know which conditions are preferred for the contacting step (b).

In step (c), the expression level of a gene (or nucleotide molecule) encoding a polypeptide as identified herein or the activity or steady state level of said polypeptide may be carried out as earlier herein defined.

In a preferred method in step (e), the difference identified in step (d) produced by the substance is a decrease of the expression level of said corresponding gene (or nucleotide molecule), or of the activity or steady state level of said polypeptide.

A decrease of the expression level of a gene (or nucleotide molecule) encoding a polypeptide as identified herein (or steady state level of said polypeptide) is preferably defined as being a detectable change of the expression level of said polypeptide and/or of a gene (or nucleotide molecule) encoding said polypeptide (or steady state level of the encoded polypeptide) or any detectable change in a biological activity of said polypeptide using a method as defined earlier on as compared to the expression level of a given polypeptide and/or of a corresponding gene (or nucleotide molecule) (or steady state level of the corresponding encoded polypeptide in a control subject. According to a preferred embodiment, a decrease of the expression level of a gene (or nucleotide molecule) encoding a given polypeptide as identified herein is quantified using a specific mRNA assay for corresponding gene as earlier defined herein. Preferably, a decrease of the expression level of a gene (or nucleotide molecule) encoding a given polypeptide means a decrease of at least 5% of the expression level of the gene (or nucleotide molecule) using PCR. Preferred primers used for the PCR are already identified herein. More preferably, a decrease of the expression level of a gene (or nucleotide molecule) means a decrease of at least 10%, even more preferably at least 20%, at least 30%, at least 40%, at least 50%, at least 70%, at least 90%, at least 150% or more.

Preferably, a decrease of the expression level of a given polypeptide means a decrease of at least 5% of the expression level of said polypeptide using western blotting and/or using ELISA or a suitable assay. More preferably, a decrease of the expression level of a polypeptide means a decrease of at least 10%, even more preferably at least 20%, at least 30%, at least 40%, at least 50%, at least 70%, at least 90%, at least 150% or more.

Preferably, a decrease of an activity of a given polypeptide means a decrease of at least 5% of the polypeptide activity using a suitable assay. More preferably, a decrease of the polypeptide activity means a decrease of at least 10%, even more preferably at least 20%, at least 30%, at least 40%, at least 50%, at least 70%, at least 90%, at least 150% or more.

In a most preferred method for identifying a substance capable of preventing, treating and/or delaying the progression of metabolic syndrome in a subject when the comparison leads to the comparison leads to a decrease of the expression level of a gene (or nucleotide molecule) as identified herein, said decrease being detected at the level of a gene (or nucleotide molecule) (a decrease of at least 5% of the expression level of the gene (or nucleotide molecule)) using PCR primers as defined herein.

Preferred genes and corresponding polypeptides have already been defined herein.

In one further aspect, the invention also pertains to a substance that is identified in a method the aforementioned methods.

### Sequence Identity

“Sequence identity” is herein defined as a relationship between two or more amino acid (polypeptide or protein) sequences or two or more nucleic acid (polynucleotide) sequences, as determined by comparing the sequences. The whole SEQ ID NO may be used or part thereof. In a preferred embodiment, the whole SEQ ID NO as identified herein is used. In the art, “identity” also means the degree of sequence relatedness between amino acid or nucleic acid sequences, as the case may be, as determined by the match between strings of such sequences. “Similarity” between two amino acid sequences is determined by comparing the amino acid sequence and its conserved amino acid substitutes of one polypeptide to the sequence of a second polypeptide. “Identity” and “similarity” can be readily calculated by known methods, including but not limited to those described in (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heine, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48:1073 (1988).

Preferred methods to determine identity are designed to give the largest match between the sequences tested. Methods to determine identity and similarity are codified in publicly available computer programs. Preferred computer program methods to determine identity and similarity between two sequences include e.g. the GCG program package (Devereux, J., et al., Nucleic Acids Research 12 (1): 387 (1984)), BestFit, BLASTP, BLASTN, and FASTA (Altschul, S. F. et al., J. Mol. Biol. 215:403-410 (1990). The BLAST X program is publicly available from NCBI and other sources (BLAST Manual, Altschul, S., et al., NCBI NLM NIH Bethesda, Md. 20894; Altschul, S., et al., J. Mol. Biol. 215:403-410 (1990). The well-known Smith Waterman algorithm may also be used to determine identity.

Preferred parameters for polypeptide sequence comparison include the following: Algorithm: Needleman and Wunsch, J. Mol. Biol. 48:443-453 (1970); Comparison matrix: BLOSSUM62 from Hentikoff and Hentikoff, Proc. Natl. Acad. Sci. USA. 89:10915-10919 (1992); Gap Penalty: 12; and Gap Length Penalty: 4. A program useful with these parameters is publicly available as the “Ogap” program from Genetics Computer Group, located in Madison, Wis. The aforementioned parameters are the default parameters for amino acid comparisons (along with no penalty for end gaps).

Preferred parameters for nucleic acid comparison include the following: Algorithm: Needleman and Wunsch, J. Mol. Biol. 48:443-453 (1970); Comparison matrix: matches=+10, mismatch=0; Gap Penalty: 50; Gap Length Penalty: 3. Available as the Gap program from Genetics Computer Group, located in Madison, Wis. Given above are the default parameters for nucleic acid comparisons.

Optionally, in determining the degree of amino acid similarity, the skilled person may also take into account so-called “conservative” amino acid substitutions, as will be clear to the skilled person. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulphur-containing side chains is cysteine and methionine. Preferred conservative amino acids substitution groups are: valine-leucine-isoleucine, phenylalanine-tyrosine, lysine-arginine, alanine-valine, and asparagine-glutamine. Substitutional variants of the amino acid sequence disclosed herein are those in which at least one residue in the disclosed sequences has been removed and a different residue inserted in its place. Preferably, the amino acid change is conservative. Preferred conservative substitutions for each of the naturally occurring amino acids are as follows: Ala to Ser; Arg to Lys; Asn to Gln or His; Asp to Glu; Cys to Ser or Ala; Gln to Asn; Glu to Asp; Gly to Pro; His to Asn or Gln; Ile to Leu or Val; Leu to Ile or Val; Lys to Arg, Gln or Glu; Met to Leu or Ile; Phe to Met, Leu or Tyr; Ser to Thr; Thr to Ser; Trp to Tyr; Tyr to Trp or Phe; and Val to Ile or Leu.

### Antibodies

Some aspects of the invention concern the use of an antibody or antibody-fragment that specifically binds to a polypeptide as identified herein. Methods for generating antibodies or antibody-fragments that specifically bind to a polypeptide are described in e.g. Harlow and Lane (1988, Antibodies: A Laboratory Manual, Cold Spring Harbor Laboratory Press, Cold Spring Harbor, N.Y.) and WO 91/19818; WO 91/18989; WO 92/01047; WO 92/06204; WO 92/18619; and U.S. Pat. No. 6,420,113 and references cited therein. The term “specific binding,” as used herein, includes both low and high affinity specific binding. Specific binding can be exhibited, e.g., by a low affinity antibody or antibody-fragment having a Kd of at least about 10⁻⁴ M. Specific binding also can be exhibited by a high affinity antibody or antibody-fragment, for example, an antibody or antibody-fragment having a Kd of at least about of 10⁻⁷ M, at least about 10⁻⁸ M, at least about 10⁻⁹ M, at least about 10⁻¹⁰ M, or can have a Kd of at least about 10⁻¹¹ M or 10⁻¹² M or greater.

### Recombinant Techniques and Methods for Over-Expression of a Polypeptide as Identified Herein in a Test Cell or in a Test Animal

A polypeptide for use in the present invention can be prepared using recombinant techniques, in which a gene (or nucleotide molecule) encoding said polypeptide of interest is (over)expressed in a suitable host cell. The present invention thus also concerns the use of a vector comprising a nucleic acid molecule as defined above. Preferably the vector is a replicative vector comprising on origin of replication (or autonomously replication sequence) that ensures multiplication of the vector in a suitable host for the vector. Alternatively a vector is capable of integrating into a host cell's genome, e.g. through homologous recombination or otherwise. A particularly preferred vector is an expression vector wherein a nucleotide molecule encoding a polypeptide as defined above, is operably linked to a promoter capable of directing expression of the coding sequence in a host cell for the vector.

As used herein, the term “promoter” refers to a nucleic acid fragment that functions to control the transcription of one or more genes, located upstream with respect to the direction of transcription of the transcription initiation site of the gene, and is structurally identified by the presence of a binding site for DNA-dependent RNA polymerase, transcription initiation sites and any other DNA sequences, including, but not limited to transcription factor binding sites, repressor and activator protein binding sites, and any other sequences of nucleotides known to one of skill in the art to act directly or indirectly to regulate the amount of transcription from the promoter. A “constitutive” promoter is a promoter that is active under most physiological and developmental conditions. An “inducible” promoter is a promoter that is regulated depending on physiological or developmental conditions. A “tissue specific” promoter is only active in specific types of differentiated cells/tissues, such as preferably a monocyte or a macrophage cell or tissue derived there from.

Expression vectors allow a polypeptide of the invention as defined above to be prepared using recombinant techniques in which a nucleotide molecule encoding said polypeptide of interest is expressed in a suitable cell, e.g. cultured cells or cells of a multicellular organism, such as described in Ausubel et al., “Current Protocols in Molecular Biology”, Greene Publishing and Wiley-Interscience, New York (1987) and in Sambrook and Russell (2001, supra); both of which are incorporated herein by reference in their entirety. Also see, Kunkel (1985) Proc. Natl. Acad. Sci. 82:488 (describing site directed mutagenesis) and Roberts et al. (1987) Nature 328:731-734 or Wells, J. A., et al. (1985) Gene 34: 315 (describing cassette mutagenesis).

Typically, a nucleotide molecule encoding a desired polypeptide is used in an expression vector. The phrase “expression vector” generally refers to a nucleotide molecule represented by a nucleotide sequence that is capable of effecting expression of a gene in hosts compatible with such sequences. These expression vectors typically include at least suitable promoter sequences and optionally, transcription termination signals. Additional factors necessary or helpful in effecting expression can also be used as described herein. A nucleic acid or DNA encoding a polypeptide is incorporated into a DNA construct capable of introduction into and expression in an in vitro cell culture. Specifically, DNA constructs are suitable for replication in a prokaryotic host, such as bacteria, e.g., E. coli, or can be introduced into a cultured mammalian, plant, insect, e.g., Sf9, yeast, fungi or other eukaryotic cell lines.

DNA constructs prepared for introduction into a particular host typically include a replication system recognized by the host, the intended DNA segment encoding the desired polypeptide, and transcriptional and translational initiation and termination regulatory sequences operably linked to the polypeptide-encoding segment. A DNA segment is “operably linked” when it is placed into a functional relationship with another DNA segment. For example, a promoter or enhancer is operably linked to a coding sequence if it stimulates the transcription of the sequence. DNA for a signal sequence is operably linked to DNA encoding a polypeptide if it is expressed as a pre-protein that participates in the secretion of the polypeptide. Generally, DNA sequences that are operably linked are contiguous, and, in the case of a signal sequence, both contiguous and in reading phase. However, enhancers need not be contiguous with the coding sequences whose transcription they control Linking is accomplished by ligation at convenient restriction sites or at adapters or linkers inserted in lieu thereof.

The selection of an appropriate promoter sequence generally depends upon the host cell selected for the expression of the DNA segment. Examples of suitable promoter sequences include prokaryotic, and eukaryotic promoters well known in the art (see, e.g. Sambrook and Russell, 2001, supra). The transcriptional regulatory sequences typically include a heterologous enhancer or promoter that is recognised by the host. The selection of an appropriate promoter depends upon the host, but promoters such as the trp, lac and phage promoters, tRNA promoters and glycolytic enzyme promoters are known and available (see, e.g. Sambrook and Russell, 2001, supra). Expression vectors include the replication system and transcriptional and translational regulatory sequences together with the insertion site for the polypeptide encoding segment can be employed. Examples of workable combinations of cell lines and expression vectors are described in Sambrook and Russell (2001, supra) and in Metzger et al. (1988) Nature 334: 31-36. For example, suitable expression vectors can be expressed in, yeast, e.g. S. cerevisiae, e.g., insect cells, e.g., Sf9 cells, mammalian cells, e.g., CHO cells and bacterial cells, e.g., E. coli. The host cells may thus be prokaryotic or eukarotic host cells. A host cell may be a host cell that is suitable for culture in liquid or on solid media. A host cell is preferably used in a method for producing a polypeptide of the invention as defined above or in a method for identification of a substance as defined herein. Said method comprises the step of culturing a host cell under conditions conducive to the expression of a polypeptide. Optionally the method may comprise recovery of a polypeptide. A polypeptide may e.g. be recovered from the culture medium by standard protein purification techniques, including a variety of chromatography methods known in the art per se.

Alternatively, a host cell is a cell that is part of a multi-cellular organism such as a transgenic plant or animal, preferably a non-human animal. A transgenic plant comprises in at least a part of its cells a vector as defined above. Methods for generating transgenic plants are e.g. described in U.S. Pat. No. 6,359,196 and in the references cited therein. Such transgenic plant or animal may be used in a method for producing a polypeptide of the invention as defined above and/or in a method for identification of a substance both as defined herein. For transgenic plant, the method comprises the step of recovering a part of a transgenic plant comprising in its cells the vector or a part of a descendant of such transgenic plant, whereby the plant part contains said polypeptide, and, optionally recovery of said polypeptide from the plant part. Such methods are also described in U.S. Pat. No. 6,359,196 and in the references cited therein. Similarly, the transgenic animal comprises in its somatic and germ cells a vector as defined above. The transgenic animal preferably is a non-human animal. More preferably, a non-human animal is a mouse. Methods for generating transgenic animals are e.g. described in WO 01/57079 and in the references cited therein. Such transgenic animals may be used in a method for producing a polypeptide as defined herein, said method comprising the step of recovering a body fluid from a transgenic animal comprising the vector or a female descendant thereof, wherein the body fluid contains said polypeptide, and, optionally recovery of said polypeptide from the body fluid. Such methods are also described in WO 01/57079 and in the references cited therein. The body fluid containing said polypeptide preferably is blood or more preferably milk.

Another method for preparing a polypeptide is to employ an in vitro transcription/translation system. DNA encoding a polypeptide is cloned into an expression vector as described supra. The expression vector is then transcribed and translated in vitro. The translation product can be used directly or first purified. A polypeptide resulting from in vitro translation typically does not contain the post-translation modifications present on polypeptides synthesised in vivo, although due to the inherent presence of microsomes some post-translational modification may occur. Methods for synthesis of polypeptides by in vitro translation are described by, for example, Berger & Kimmel, Methods in Enzymology, Volume 152, Guide to Molecular Cloning Techniques, Academic Press, Inc., San Diego, Calif., 1987.

In this document and in its claims, the verb “to comprise” and its conjugations is used in its non-limiting sense to mean that items following the word are included, but items not specifically mentioned are not excluded. In addition the verb “to consist” may be replaced by “to consist essentially of” meaning that a polypeptide or a nucleic acid construct or an antibody as defined herein may comprise additional component(s) than the ones specifically identified, said additional component(s) not altering the unique characteristic of the invention. In addition, reference to an element by the indefinite article “a” or “an” does not exclude the possibility that more than one of the element is present, unless the context clearly requires that there be one and only one of the elements. The indefinite article “a” or “an” thus usually means “at least one”. Each embodiment as identified herein may be combined together unless otherwise indicated. All patent and literature references cited in the present specification are hereby incorporated by reference in their entirety.

The invention is further illustrated by the following examples which should not be construed for limiting the scope of the present invention.

### DESCRIPTION OF THE FIGURES

FIG. 1. Body weight and oral glucose tolerance test. (A) Body weight gain of C57BL/6J mice during a low-fat or high-fat diet intervention of 8 weeks. (B) and (C) An oral glucose tolerance test was performed after 7 weeks of diet intervention. After an oral gavage of 100 mg glucose, blood glucose levels were monitored for 150 minutes. The changes in blood glucose levels (B) and the area under the curve were calculated (C). In (A) and (B), data are means±SEM.*p<0.05. LF=low-fat diet, HF=high-fat diet.

FIG. 2. Dietary fat-induced differential gene expression along the longitudinal axis of the small intestine. For the proximal (SI 1), middle (SI 2) and distal part of the small intestine (SI 3), the numbers of genes that are differentially expressed in at least one week of diet intervention are plotted (grey bars). Among those genes are genes that are consistently up- (I) or down-regulated (D) on a high-fat diet (white and black bars, respectively).

FIG. 3. Immunohistochemical analysis of dietary fat-induced cell proliferation in the small intestine of C57BL/6J mice. Immunohistochemistry was performed on distal small intestinal sections of C57BL/6J mice fed a low-fat (A) or high-fat diet (B) using Ki67-specific antibodies. The villus is defined from dotted line to top of the villus and the arrow indicates Ki67-specific staining (brown) at the bottom of the villus. Next to the number of Ki67-positive cells per villus (C), also the total number of villus cells (D) and villus length (E) were determined. Therefore, per mouse 15 villi were observed and the mean values were calculated. A-specific staining was detectable in the lamina propria due to cross-reactivity of the goat-anti-rat antibody (also seen in negative control without Ki67 antibodies, data not shown). *p<0.05. LF=low-fat diet, HF=high-fat diet.

FIGS. 4 and 5. Heat map diagrams of differentially expressed genes on a high-fat diet. SLR of differentially expressed genes related to lipid metabolism (A), cell cycle (B) and inflammation/immune response (C) are clustered in a heat map diagram for the proximal (SI 1), middle (SI 2) and distal part of the small intestine (SI 3). FIG. 4 relates to the down-regulation of gene expression, whereas FIG. 5 relates to the up-regulation. Amongst other genes that display similar expression patterns on a high-fat diet, the boxes include differentially expressed genes that share association with particular biological processes (numbered). Differentially expressed genes with a −0.3>SLR>0.3 in at least one week of diet intervention are included and the color scheme ranges from SLR −1.5 to 1.5. Next to SLR, also the numbers of differentially expressed genes are visualized.

FIG. 6. Verification of microarray results in individual mice by qPCR analysis. For the proximal (A), middle (B) and distal part of the small intestine (C), five genes that were found to be differentially expressed by microarray analysis were randomly selected and their expression was validated in individual mouse samples by qPCR. Only the results of the 18S normalization are shown as they are representative for the results of the cyclophilin A normalization. The qPCR data are visualized as the mean expression of all individual mice per diet group per time point ±SEM, relative to the expression on the LF diet at week 2 which was set to 1.*p<0.05. LF=low-fat diet, HF=high-fat diet.

### EXAMPLES

### Materials & Methods

### Animals and Diets

Male C57BL/6J mice were purchased from Harlan (Horst, The Netherlands) and were housed in the light- and temperature-controlled animal facility of Wageningen University. They had free access to water and prior to the diet intervention they received standard laboratory chow (RMH-B, Arie Blok BV, Woerden, The Netherlands). All experiments were approved by the Ethical Committee on animal testing of Wageningen University.

In this study we investigated the effect of dietary fat on development of obesity and insulin resistance and on small intestinal gene expression in C57BL/6J mice. After a run-in period of 3 weeks on the low-fat diet, 9 week old mice were fed a powder high- or a low-fat purified diet for 2, 4, and 8 weeks (n=6 per diet, per time point). Low-fat and high-fat diets are based on ‘Research Diets’ formulas D12450B/D12451, with adaptations regarding type of fat (palm oil in stead of lard) and carbohydrates to mimic the fatty acid and carbohydrate composition of the average human diet in Western societies (Research diet services, Wijk bij Duurstede, The Netherlands). The complete compositions of the diets are given in supplementary table S1. It should be noted that in these diets the energy density of all nutrients, except fat and starch, are equal. Body weight was recorded weekly and after 7 weeks of diet intervention an oral glucose tolerance test was performed. Therefore, after 6-hours fasting, mice received 0.5 ml of a 20% glucose solution via an oral gavage and blood glucose was measured after 15, 30, 45, 60, 90 and 150 minutes using Accu-Chek blood glucose meters (Roche Diagnostics, Almere, The Netherlands). At the end of the experiment, mice were anaesthetized with a mixture of isofluorane (1.5%), nitrous oxide (70%) and oxygen (30%). The small intestines were excised and the adhering fat and pancreatic tissue were carefully removed. The small intestines were divided in three equal parts along the proximal to distal axis (SI 1=proximal part; duodenum, SI 2=middle part; jejunum and SI 3=distal part; ileum). Small intestinal epithelial cells were scraped, snap-frozen in liquid nitrogen, and stored at −80° C. until RNA isolation. For immunohistochemical analysis, a similar low-fat and high-fat diet intervention study was performed for 2 weeks (n=12 per diet). Small intestines were again excised, divided in three equal parts, cut open longitudinally, and washed with PBS. Thereafter, the small intestinal parts were fixed in 10% buffered formalin and imbedded in paraffin as ‘Swiss rolls’.

### RNA Isolation

Total RNA was isolated using TRIzol reagent (Invitrogen, Breda, The Netherlands) according to the manufacturer's instructions. The isolated RNA was further column-purified using the SV total RNA isolation system (Promega, Leiden, The Netherlands). RNA concentration was measured on a Nanoprop ND-1000 UV-Vis spectrophotometer (Isogen, Maarssen, The Netherlands) and analyzed on a bioanalyzer (Agilent Technologies, Amsterdam, the Netherlands) with 6000 Nano Chips according to the manufacturer's instructions.

### Microarray Hybridization and Analysis

For each part of the small intestine, total RNA was pooled per diet group and per time point (n=6). RNA was hybridized to Mouse genome 430 2.0 arrays (Affymetrix, Santa Clara, Calif., USA). Detailed methods for the labelling and subsequent hybridizations to the arrays are described in the eukaryotic section in the GeneChip Expression Analysis Technical Manual Rev. 3 from Affymetrix. Arrays were scanned on an Affymetrix GeneChip Scanner 3000. Data analysis was performed using Microarray Analysis Suite 5.0 (MAS 5.0). To estimate the magnitude and direction of differential gene expression for the high-fat versus low-fat treatments, MAS 5.0 software provides signal log ratio's (SLR). If the SLR is equal to or greater than 0, fold change is obtained with +2^{SLR}, otherwise with −2^{-SLR}. Array data have been submitted to the Gene Expression Omnibus, accession number GSE8582.

To determine overrepresentation of Gene Ontology (GO) Biological Process subsets upon high-fat diet intervention, an ErmineJ overrepresentation analysis (ORA) was performed (33). Gene score files that were used as input contained the ‘change p-values’ of all probes sets provided by MAS 5.0 comparison analysis. By setting the Gene score threshold to 0.0025, only significantly differentially expressed genes were included in the ORA analyses. Moreover, only GO subsets that contained between 2 and 125 genes were taking into account. The ErmineJ software generally uses Benjamini-Hochberg correction of p-values to determine which gene sets are selected with a particular false discovery rate (FDR). The FDR is considered a rapid and reasonable guide to which gene sets are likely to be of highest interest.

Heat map diagrams visualizing SLR of differentially expressed genes are made using Spotfire DecisionSite® software by applying hierarchical clustering.

cDNA Synthesis and Real-Time Quantitative PCR

Single-stranded complementary DNA (cDNA) was synthesized from 1 μg of total RNA using the Reverse transcription system (Promega, Leiden, The Netherlands) following the supplier's protocol. cDNA was PCR amplified with Platinum Taq DNA polymerase (all reagents were from Invitrogen). Primer sequences that we used for real-time quantitative PCR reaction (qPCR) were chosen based on the sequences available in the GenBank database (www.ncbi.nlm.nih.gov) and these are listed in supplementary table S2. qPCRs was performed using SYBR green and a MyIQ thermal cycler (Bio-Rad laboratories BV, Veenendaal, The Netherlands). The following thermal cycling conditions were used: 2 min at 94° C., followed by 40 cycles of 94° C. for 15 s and 60° C. for 45 s. PCR reactions were performed in duplicate and all samples were normalized to 18S and cyclophilin A expression.

### Immunohistochemistry

Four-micrometer sections of paraffin-embedded distal part of the small intestine were mounted on Superfrost microscope slides. These sections were dewaxed in xylene and rehydrated in a series of graded alcohols. To block endogenous peroxidase activity, slides were incubated with 3% H₂O₂ for 20 minutes. Antigen retrieval is performed by placing the slides in citrate buffer (pH 6.0) and heat them in a microwave oven 5 min 700 W (without lid) and 4 times 5 min 500 W (with lid). After cooling down to room temperature, the sections were briefly washed with PBS. Prior to staining, a 20 minutes preincubation was performed using 20% normal goat serum (Vector Laboratories, Burlingame, Calif., USA). The sections were stained in a three-step procedure utilizing the following incubations: overnight incubation at 4° C. with rat monoclonal antibodies against Ki67 (Clone TEC-3) (DakoCytomation B.V., Heverlee, Belgium), diluted 1:200 in PBS. Thereafter, the sections were incubated with a biotinylated goat-anti-rat for 30 minutes, followed by 45 minutes incubation with peroxidase-labelled avidin-biotin complex (Vector Laboratories). Between all incubations, sections were washed three times in PBS. Diaminobenzidine tetrahydrochloride (DAB, Vector Laboratories) was used as substrate to visualize the bound antibodies. After counterstaining with Meyer's hematoxylin, sections were mounted with DePex mounting medium (Gurr, BDH, Poole, Dorset, UK).

### Statistical Analysis

All data are reported as the mean±SEM. The differences between the mean values were tested for statistical significance by the two-tailed Student's t test.

### Results

### Dietary Fat-Induced Obesity and Insulin Resistance in C57BL/6J Mice

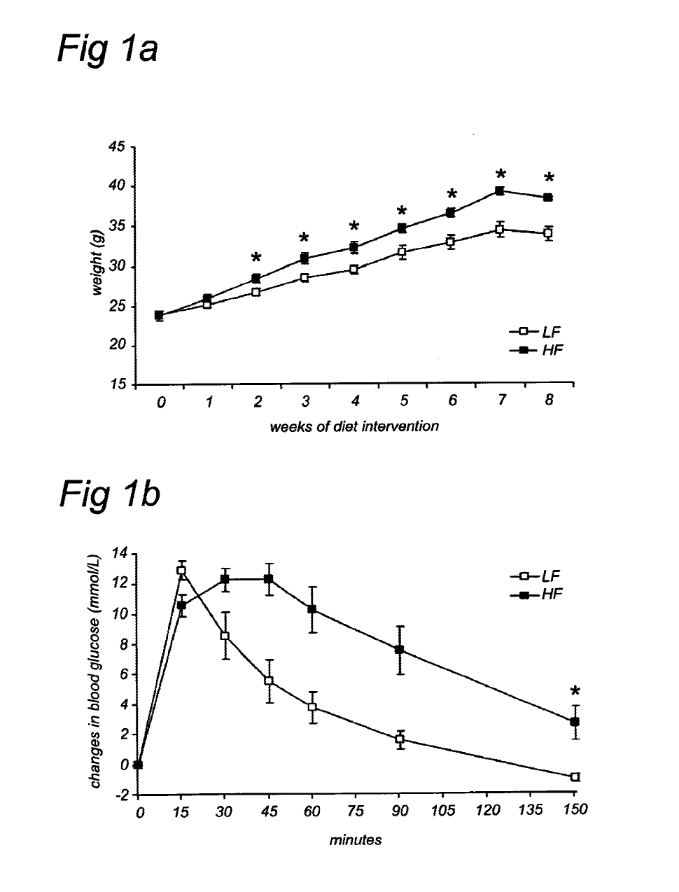

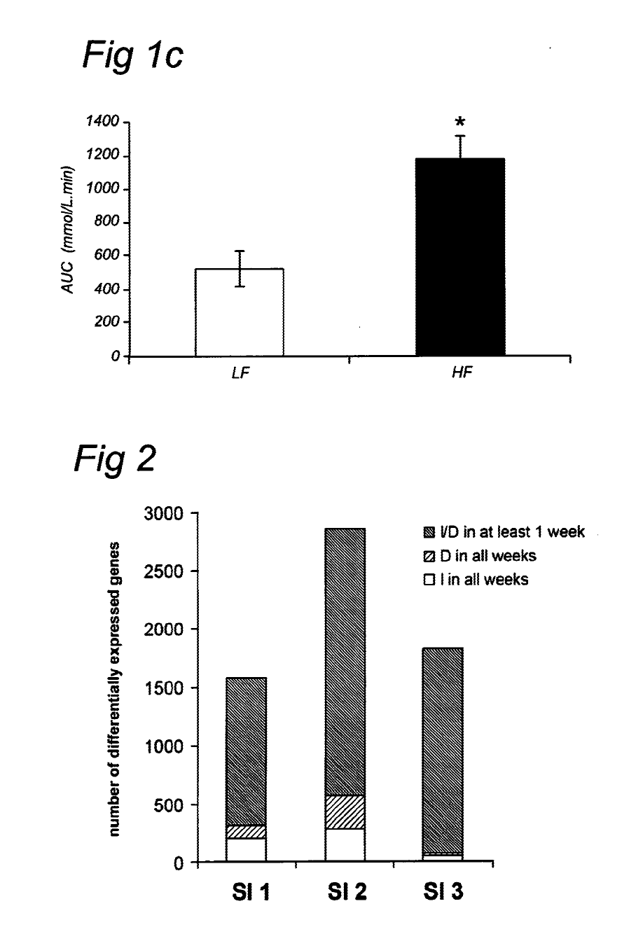

To evaluate the effect of a high-fat diet on the development of obesity and insulin resistance, C57BL/6J mice were fed a high-fat versus low-fat diet for eight weeks. FIG. 1A shows that already after two weeks on the high-fat diet, C57BL/6J mice demonstrate a significantly higher weight gain than mice on the low-fat diet. Moreover, an oral glucose tolerance test performed after seven weeks of diet intervention showed that the high-fat diet induced a significantly higher glucose intolerance (FIG. 1B), indicating development of insulin resistance. As the intake of the high-fat and low-fat diets was isoenergetic, these data indicate that dietary fat (palm oil) induces obesity and insulin resistance in C57BL/6J mice.

### Dietary Fat-Induced Changes in Small Intestinal Gene Expression

After 2, 4 and 8 weeks of diet intervention, C57BL/6J mice were sacrificed (n=6 per diet, per time point), small intestines were isolated and divided into three equal parts along the proximal to distal axis. For each part of the small intestine, dietary fat-induced differential gene expression was analyzed and the numbers of genes that are differentially expressed in at least one week of diet intervention are visualized in FIG. 2. The consistently differentially expressed genes with a fold change lower than −3 and higher than +3 are listed in supplementary table S3. The highest numbers of genes, transiently as well as sustained differentially expressed during diet intervention, are found in the middle part of the small intestine, indicating that the effect of dietary fat on gene expression is most pronounced in the jejunum.

As microarray analyses in this study were performed on mouse samples that were pooled per diet group per time point, qPCR was used to verify differential gene expression of randomly selected genes in individual mouse samples. qPCR results were highly in accordance with the microarray data (supplementary figure S1), especially in the middle part of the small intestine. In the proximal and distal part of the small intestine, differential expression of some genes did not reach significance by qPCR, due to a higher variance among individual mouse samples.

### Biological Processes Influenced by Dietary Fat in the Small Intestine

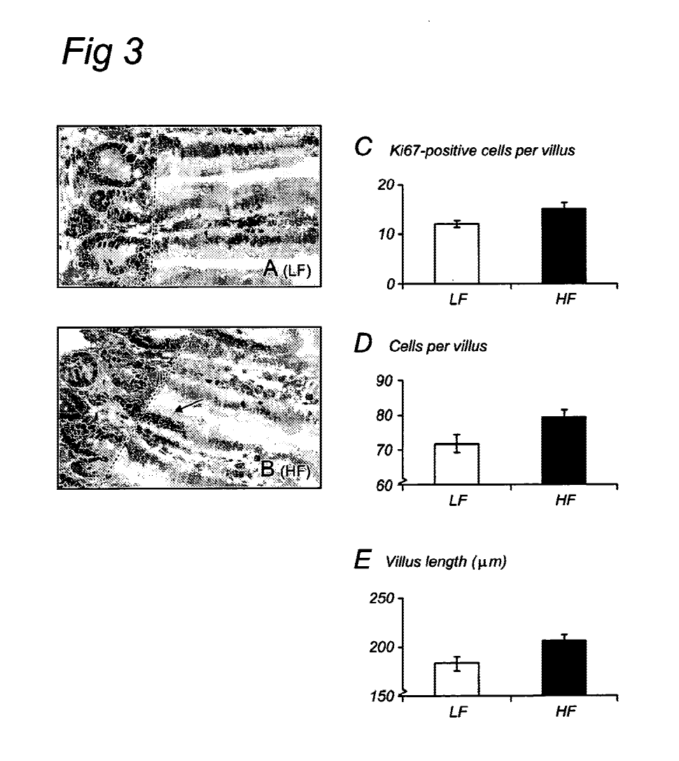

To determine which biological processes in the small intestine are highly influenced by dietary fat, we performed an overrepresentation analysis (ORA) for each part of the small intestine, including all genes showing differential expression in at least one week of diet intervention. The false discovery rate (FDR) that is calculated in the ORA analysis is considered a rapid and reasonable guide to which gene sets are likely to be of highest interest (33). Table 1 displays the GO Biological Process subsets that are overrepresented in the different parts of the small intestine (FDR <0.01) in mice fed a high-fat diet. Additionally, in FIG. 3, heat map diagrams illustrate the numbers of genes that are annotated to the GO terms listed in table 1 and the magnitude and direction of their differential gene expression indicated by the SLR.

ORA analysis reveals that biological processes related to lipid metabolism are highly regulated by dietary fat in all parts of the small intestine. Additionally, the heat map diagrams in FIG. 3A show that the up- or down-regulation of many lipid metabolism related genes is very consistent in time and that the strongest dietary fat effects can be observed in the proximal and middle part of the small intestine. In these parts, genes involved in fatty acid transport, chylomicron synthesis and especially fatty acid oxidation are highly up-regulated, whereas genes involved in cholesterol transport are down-regulated by dietary fat. In the distal part of the small intestine, similar regulation of fatty acid oxidation and cholesterol transport is seen, but less prominent than in the more proximal parts of the small intestine. These data indicate that dietary fat processing/handling is mainly accomplished in the duodenum and jejunum. However, the ileum is still capable of handling the overflow of fat.

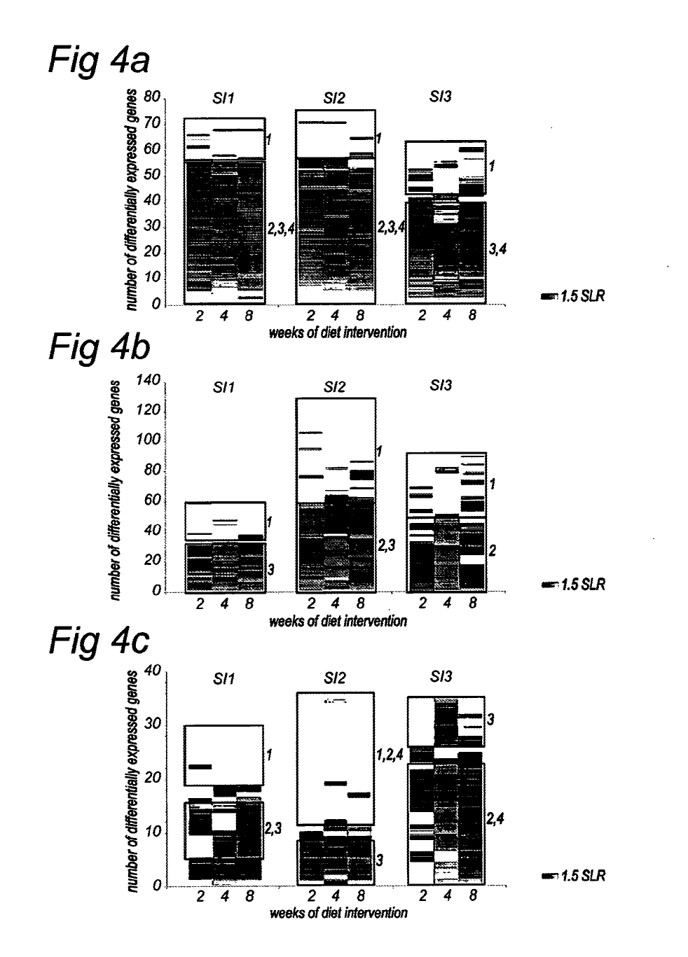

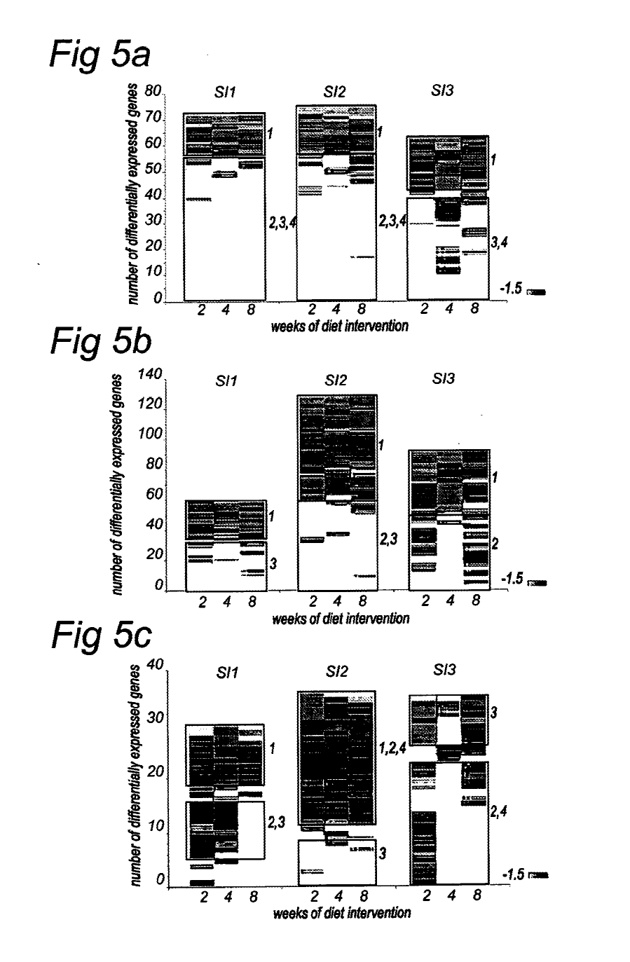

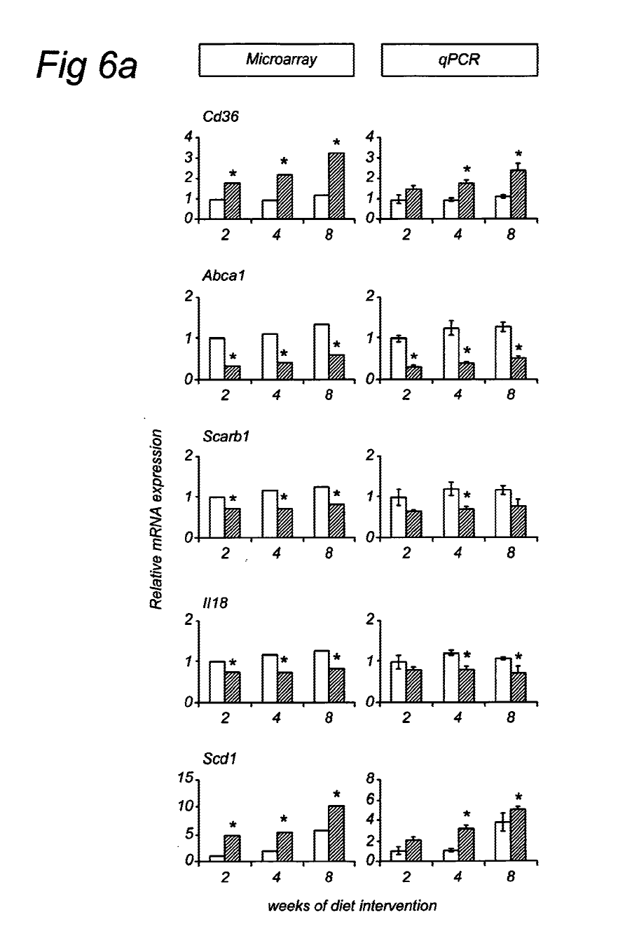

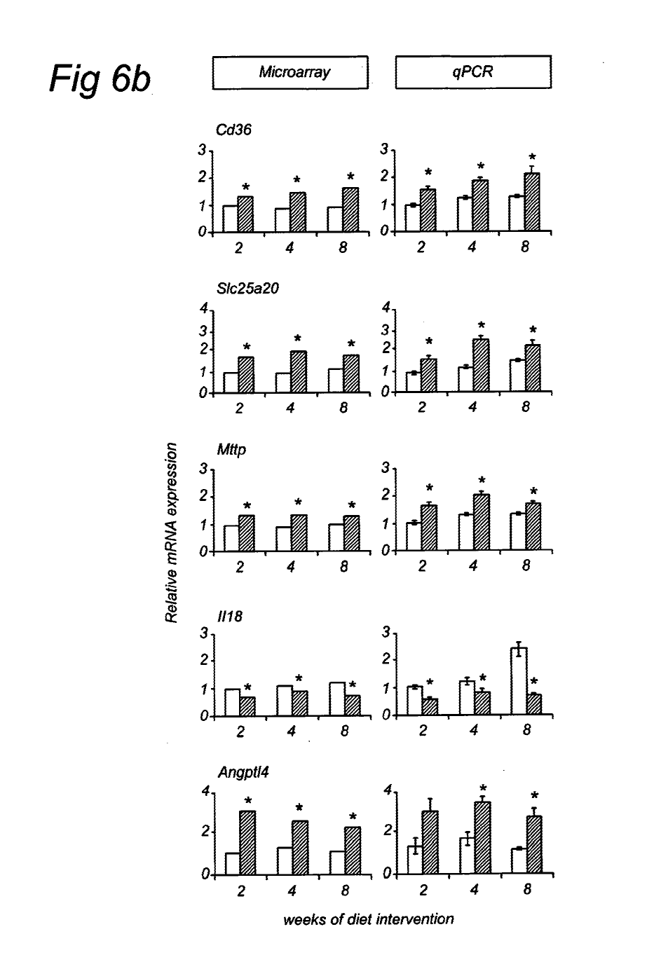

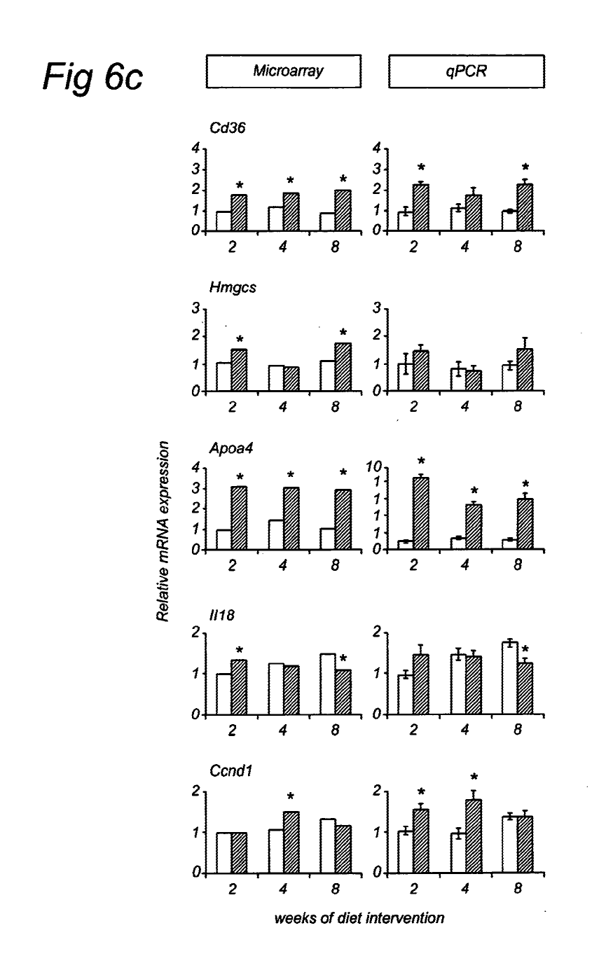

Additionally, ORA analysis and heat map diagrams (FIG. 3B) show the effect of dietary fat on regulation of cell cycle related processes, which is most pronounced in the middle and distal parts of the small intestine. Cell proliferation seems to be enhanced by dietary fat early in diet intervention, as genes that are essential for progression through cell cycle are up-regulated in the first weeks and genes involved in apoptosis are down-regulated. Remarkably, after 8 weeks of diet intervention, hardly any differential gene expression related to cell cycle can be detected. To ensure that the dietary fat-induced modulations in cell cycle related processes reflect proliferation of enterocytes and not immune cells, we performed immunohistochemistry on the distal part of the small intestine using Ki67-specific antibodies (FIG. 4). Differences in Ki67-staining of the small intestines exposed to the low-fat or high-fat diet were most pronounced at the bottom of the villi (FIGS. 4A and 4B). Although this increase in Ki67-positive cells per villus did not reach significance (p=0.07), villus length and the total number of cells per villus were significantly higher in mice fed the high-fat diet. These data indicate that cell proliferation induced by dietary fat results in enlargement of small intestinal villi.

Biological Process subsets related to inflammation/immune response are also overrepresented in small intestine after feeding a high-fat diet. In the proximal and distal small intestine, the differential expression of genes related to these processes is not very consistent in time. However, in the middle part of the small intestine, a substantial number of genes show a sustained down-regulation throughout high-fat diet intervention. Remarkably, many of these down-regulated genes are known to be interferon gamma (IFNγ)-inducible genes (28, 42). Despite this consistent down-regulation of genes in the jejunum, which suggests a diminished inflammatory disposition after exposure to elevated levels of dietary fat, it remains difficult to draw a definitive conclusion on inflammatory status of the overall small intestine, as hardly any gene shows a consistent up- or down-regulation in all parts of the small intestine. Taken together, ORA analysis showed that dietary fat highly influences processes related to lipid metabolism, proliferation and inflammation and/or immune response in the small intestine of C57Bl/6J mice.

### Dietary Fat-Induced Gene Expression Changes of Small Intestinal Secreted Proteins

In response to dietary components, the small intestinal mucosa can be triggered to secrete signaling proteins that are able to induce systemic effects, such as modulation of metabolism in peripheral organs. To identify secreted proteins that are differentially expressed during high-fat diet intervention, we performed a secretome analysis. For this analysis, genes that were differentially expressed in at least one week of diet intervention (fold change >1.5) were additionally selected for their corresponding GO Cellular Component term ‘extracellular region/space’ (GO:0005576/GO:0005615) (Table 2). Some of these selected genes showed a consistent differential gene expression throughout the small intestine (e.g. Angptl4, ApoC2, Dnase1, Cgref1, Gas6, H2-Q10), whereas for other genes the changes were more restricted to a particular part of the gut (e.g. Cck, Igfbp3, Reg1, Fgf15, Ccl28, Ccl5, Pyy). For several genes, also a time effect could be detected, as they were showing early (e.g. Igfbp4, Ttr) or late phase (e.g. Ccl5, Ccl28, Igj, Fgf15) responses. Consistent with the ORA analysis data described above, many of the secreted proteins are related to lipid metabolism, especially chylomicron synthesis (e.g. ApoA4, ApoC2, ApoC3), and inflammation/immune response (e.g. several chemokines, H2-Q10, I118, Mif, Rsad2, Saa1/2). Although we do not propose that all of these secreted proteins act as signaling molecules that provoke a systemic effect on peripheral organs, we consider genes related to inflammation/immune response and the gut hormones (e.g. Cck, Pyy) as promising candidates. Also genes with a pronounced differential gene expression, of which function is not completely elucidated yet (e.g. Angptl4, Oit1, Smpdl3a/b) are potential interesting signaling molecules that might contribute to development of obesity and/or insulin resistance. Table 4 identifies the same genes as table 2, gives their tissue distribution and their level of expression. In grey in table 4, 15 genes are selected for their high level of expression in the animal model and their restricted tissue distribution (restricted till specific gastro-intestinal tract). Each of these genes alone or in combination are attractive to be used as markers in the present invention since their gene product is secreted into the serum, their expression level is up-regulated in the studied animal model and they are expressed in a limited number of tissues. The 15 genes of Table 4 are further identified in Table 5. As Fam3D (Oit1) and ApoA4 are gut-specific markers (small intestine), their differences in gene expression as measured in serum may easily be extrapolated to differences in gene expression in the small intestine.

### Discussion

In this study, we demonstrated that C57BL/6J mice develop obesity and glucose intolerance on a high-fat diet that mimics the fatty acid composition of a Western-style human diet. Microarray analysis showed that dietary fat induces a substantial number of changes in gene expression throughout the small intestine. However, the most pronounced effects were detectable in the middle part of the small intestine. Biological processes that we found to be highly influenced in the small intestine by feeding a high-fat diet are predominantly associated with lipid metabolism, inflammation/immune response and cell cycle.

Lipid metabolism related genes, especially Ppara target genes involved in fatty acid transport and fatty acid oxidation, were highly and consistently regulated by dietary fat. This indicates that lipid metabolism related processes are presumably very important for efficient dietary fat handling in the small intestine. Kondo et al. recently compared the gene expression levels of several Pparα target genes involved in fatty acid catabolism between obesity-resistant A/J versus obesity-prone C57Bl/6J mice after feeding a high-fat diet (27). In their study, the basal as well as the dietary fat-induced up-regulated expression of the genes were higher in the A/J mice compared to the C57Bl/6J mice. They suggested that in C57Bl/6J mice fatty acid catabolism in the small intestine proceeds less efficient than in A/J mice and that an impaired activation of Ppara might play an important role in this process. Moreover, the highly reduced expression level of Cyp4a10 in C57Bl/6J mice suggested that ω-oxidation plays an essential role in the diminished efficacy of small intestinal fatty acid handling. The co-oxidation is known to be a compensatory mechanism when β-oxidation is not sufficient, which seems to be the case on a high-fat diet intervention. Interestingly, it was previously shown that also Ppargc1a is involved in fatty acid oxidation, as together with Ppara it can cooperatively induce the expression of Ppara target genes and increase cellular fatty acid oxidation rates (49). Moreover, a decreased expression of Ppargc1a was linked to an inefficient fatty acid oxidation and associated with an impaired glucose tolerance in mice fed a high-fat diet (29). Based on these studies, we speculate that in the C57Bl/6J mice the dietary fat-induced down-regulation of Ppargc1a is related to a suboptimal activation of Ppara. This results in an inefficient fatty acid oxidation in the small intestine, in which we believe ω-oxidation has a pronounced role. How this impaired fatty acid handling in the small intestine might contribute to development of dietary fat-induced obesity and/or insulin resistance is not yet known and has to be investigated in future studies.

Next to a role in lipid handling, Ppars are also known to be related to inflammation and immune response. Activated Ppars can suppress production of pro-inflammatory cytokines or related mediators, such as tumor necrosis factor α (Tnfα) (20), IFNγ(18) and nuclear factor kappa B (Nfkb) (10). So, lipids can regulate inflammatory and immune processes via Ppars and this might explain the down-regulation of IFNγ-inducible genes in the middle part of the small intestine, which was found to be most susceptible to dietary fat-induced gene expression changes. On the other hand, the down-regulation of IFNγ-inducible genes can be the result of the decreased expression of pro-inflammatory cytokine I118, which actions are mediated by IFNγ. Interestingly, a recent study showed that I118 null mice have markedly increased body weight and are insulin resistant (39). It is even suggested that I118 possesses a glucose-lowering potential. Based on our data, we hypothesize that this recently proposed role of I118 in obesity and insulin resistance is mediated via the IFNγ signaling pathway.

There is growing evidence that chronic inflammation contributes to development of obesity and insulin resistance (21). Some of these inflammatory pathways, which are most extensively studied in liver, adipose tissue and muscle, involve toll-like receptor 4 (Tlr4) (44), tumor necrosis factor α (Tnfα) (22), nuclear factor kappa B (Nfkb) (5), Jun kinases (Jnk) and insulin receptor substrates (Irs) (22, 47). Disturbances in these pathways can lead to disruption of insulin action/signaling and thereby affecting insulin sensitivity. However, as insulin signaling is not very likely in the small intestine, due to lack of insulin receptors, these inflammation pathways are not expected to contribute to the role of the small intestine in development of obesity and insulin resistance. In our microarray data, we could indeed not detect any expression of Tnfα, Tlr4 and Irs and no differential gene expression of Nfkrβ and Jun kinases.

Furthermore, our data showed that in the first weeks of high-fat diet intervention, cell proliferation is enhanced in the middle and distal part of the small intestine, leading to an increase in villus cell number and villus length. Petit et al, recently also reported enhanced proliferation in jejunum after feeding mice a high-fat diet, even though their diet had a somehow different fatty acid composition than was used in our study (40). The dietary fat-induced enlargement of the villi might be functional to extent the capacity of fat absorption. Remarkably, we found that the dietary fat-induced cell proliferation was attenuated after a longer period of diet intervention. This suggests that the increased uptake capacity reaches a certain maximum between 4 and 8 weeks on a high-fat diet, which might finally result in an inefficient absorption and processing of dietary fat. Although hardly any gene related to cell cycle in the small intestine was previously described to be associated with obesity and/or insulin resistance, our data indicate that small intestinal cell proliferation is important for an optimal functioning of the small intestine when exposed to a high-fat diet.

As signaling molecules secreted by the small intestine are able to induce systemic effects by influencing metabolic homeostasis in peripheral organs, inefficient or altered regulation of these molecules might be related to the etiology of obesity and/or insulin resistance (9, 12). Various studies describe the potential role of gut hormones in metabolic syndrome related disease states. For the incretin hormones Gip and Glp1, which can induce a systemic effect on glucose homeostasis, our data imply that elevated levels of the bioactive compounds are available on a high-fat diet. As previous studies showed that increased plasma levels of Gip and Glp1 even lead to an improved insulin sensitivity (36, 37), it is not very likely that the incretins contribute to development of obesity and glucose intolerance in our C57BL/6J mouse model. Other secreted proteins that are more likely to provide a substantial contribution to small intestinal involvement are I118, Ffgf15, Mif and Igfbp3. Their differential expression in the small intestine induced by dietary fat corroborates results of previous knock-out and over-expression studies showing association with obesity and/or insulin resistance (7, 14, 39, 45). Contradictory to previous studies showing that suppression of Angptl4 mediated by gut-microbiota was related to dietary fat-induced obesity (2), we found a sustained up-regulation of this gene in all parts of the small intestine. This implies that normal suppression of Angptl4 by gut microbiota (1) is consistently counteracted by dietary fat. As despite this persistent up-regulation of Angptl4, C57Bl/6J mice still became obese on a high fat diet, we conclude that small intestinal Angptl4 is probably not a main contributor to development of obesity. Interestingly, however, studies of Mandard et al. showing that elevated levels of Angptl4 are related to glucose intolerance might indicate that the dietary-fat induced up-regulation of Angptl4 in the small intestine can provoke a systemic effect on development of insulin resistance (35). Further research will be required to more accurately elucidate the function of Angptl4 in the small intestine and its potential involvement in metabolic syndrome.

In summary, we found that a high-fat diet that mimics the fatty acid composition of a Western-style human diet induces obesity and insulin resistance in C57BL/6J mice. The biological processes that are most apparently modulated in the small intestine by this dietary fat are related to lipid metabolism, cell cycle and inflammation/immune response. Additionally, secretome analysis revealed several secreted proteins with a modulated expression on a high fat diet that might provoke metabolic effects in liver, muscle and adipose tissue. As many of the genes, showing dietary fat-induced changes, were previously already linked to obesity and/or insulin resistance, this exploratory study provides several leads for an essential role of the small intestine in the etiology of these disease states. To narrow down the small intestinal contribution to development of metabolic syndrome, future research with a special focus on efficacy of fatty acid catabolism and function of small intestinal secreted proteins such as I118, Fgf15, Mif, Igfbp3 and Angptl4 will be done.

### REFERENCES

1. Backhed F, Ding H, Wang T, Hooper L V, Koh G Y, Nagy A, Semenkovich C F, and Gordon J I. The gut microbiota as an environmental factor that regulates fat storage. PNAS 101: 15718-15723, 2004.
2. Backhed F, Manchester J K, Semenkovich C F, and Gordon J I. From the Cover: Mechanisms underlying the resistance to diet-induced obesity in germ-free mice. PNAS 104: 979-984, 2007.
3. Bonen A, Tandon N N, Glatz J F C, Luiken J J F P, and Heigenhauser G J F. The fatty acid transporter FAT//CD36 is upregulated in subcutaneous and visceral adipose tissues in human obesity and type 2 diabetes. Int J Obes 30: 877, 2006.
4. Bunger M, van den Bosch H M, van der Meijde J, Kersten S, Hooiveld G J, and Muller M. Genome-wide analysis of PPAR{alpha} activation in murine small intestine. Physiol Genomics 2007.
5. Cai D, Yuan M, Frantz D F, Melendez P A, Hansen L, Lee J, and Shoelson S E. Local and systemic insulin resistance resulting from hepatic activation of IKK-[beta] and NF-[kappa]B. Nat Med 11: 183, 2005.
6. Campion J, Milagro F I, Fernandez D, and Martinez J A. Differential gene expression and adiposity reduction induced by ascorbic acid supplementation in a cafeteria model of obesity. J Physiol Biochem 62: 71-80, 2006.
7. Church T S, Willis M S, Priest E L, Lamonte M J, Earnest C P, Wilkinson W J, Wilson D A, and Giroir B P. Obesity, macrophage migration inhibitory factor, and weight loss. Int J Obes (Lond) 29: 675-681, 2005.
8. Conarello S L, Li Z, Ronan J, Roy R S, Zhu L, Jiang G, Liu F, Woods J, Zycband E, Moller D E, Thornberry N A, and Zhang B B. Mice lacking dipeptidyl peptidase IV are protected against obesity and insulin resistance. PNAS 100: 6825-6830, 2003.
9. Cummings D E, and Overduin J. Gastrointestinal regulation of food intake. J Clin Invest 117: 13-23, 2007.
10. Delerive P, Fruchart J C, and Staels B. Peroxisome proliferator-activated receptors in inflammation control. J Endocrinol 169: 453-459, 2001.
11. Drucker D J. Enhancing the Action of Incretin Hormones: A New Whey Forward? Endocrinology 147: 3171-3172, 2006.
12. Drucker D J. The role of gut hormones in glucose homeostasis. J Clin Invest 117: 24-32, 2007.
13. Duran-Sandoval D, Cariou B, Fruchart J-C, and Staels B. Potential regulatory role of the farnesoid X receptor in the metabolic syndrome. Biochimie 87: 93, 2005.
14. Fu L, John L M, Adams S H, Yu X X, Tomlinson E, Renz M, Williams P M, Soriano R, Corpuz R, Moffat B, Vandlen R, Simmons L, Foster J, Stephan J-P, Tsai S P, and Stewart T A. Fibroblast Growth Factor 19 Increases Metabolic Rate and Reverses Dietary and Leptin-Deficient Diabetes. Endocrinology 145: 2594-2603, 2004.
15. Gertow K, Pietilainen K H, Yki-Jarvinen H, Kaprio J, Rissanen A, Eriksson P, Hamsten A, and Fisher R M. Expression of fatty-acid-handling proteins in human adipose tissue in relation to obesity and insulin resistance. Diabetologia 47: 1118-1125, 2004.
16. Guerre-Millo M, Gervois P, Raspe E, Madsen L, Poulain P, Derudas B, Herbert J-M, Winegar D A, Willson T M, Fruchart J-C, Berge R K, and Staels B. Peroxisome Proliferator-activated Receptor alpha Activators Improve Insulin Sensitivity and Reduce Adiposity. J Biol Chem 275: 16638-16642, 2000.
17. Gutierrez-Juarez R, Pocai A, Mulas C, Ono H, Bhanot S, Monia B P, and Rossetti L. Critical role of stearoyl-CoA desaturase-1 (SCD1) in the onset of diet-induced hepatic insulin resistance. J Clin Invest 116: 1686-1695, 2006.
18. Hara Y, Miura S, Komoto S, Inamura T, Koseki S, Watanabe C, Hokari R, Tsuzuki Y, Ogino T, Nagata H, Hachimura S, Kaminogawa S, and Ishii H. Exposure to fatty acids modulates interferon production by intraepithelial lymphocytes. Immunol Lett 86: 139-148, 2003.
19. Herder C, Kolb H, Koenig W, Haastert B, Muller-Scholze S, Rathmann W, Holle R, Thorand B, and Wichmann H E. Association of Systemic Concentrations of Macrophage Migration Inhibitory Factor With Impaired Glucose Tolerance and Type 2 Diabetes: Results from the Cooperative Health Research in the Region of Augsburg, Survey 4 (KORA S4). Diabetes Care 29: 368-371, 2006.
20. Hill M R, Clarke S, Rodgers K, Thornhill B, Peters J M, Gonzalez F J, and Gimble J M. Effect of Peroxisome Proliferator-Activated Receptor Alpha Activators on Tumor Necrosis Factor Expression in Mice during Endotoxemia. Infect Immun 67: 3488-3493, 1999.
21. Hotamisligil G S. Inflammation and metabolic disorders. Nature 444: 860-867, 2006.
22. Hotamisligil G S, Peraldi P, Budavari A, Ellis R, White M F, and Spiegelman B M. IRS-1-Mediated Inhibition of Insulin Receptor Tyrosine Kinase Activity in TNF-alpha- and Obesity-Induced Insulin Resistance. Science 271: 665-670, 1996.
23. Jiang T, Wang Z, Proctor G, Moskowitz S, Liebman S E, Rogers T, Lucia M S, Li J, and Levi M. Diet-induced Obesity in C57BL/6J Mice Causes Increased Renal Lipid Accumulation and Glomerulosclerosis via a Sterol Regulatory Element-binding Protein-1c-dependent Pathway. J Biol Chem 280: 32317-32325, 2005.
24. Kadowaki T, Hara K, Yamauchi T, Terauchi Y, Tobe K, and Nagai R. Molecular Mechanism of Insulin Resistance and Obesity. Experimental Biology and Medicine 228: 1111-1117, 2003.
25. Kahn B B, and Flier J S. Obesity and insulin resistance. J Clin Invest 106: 473-481, 2000.
26. Kalaany N Y, Gauthier K C, Zavacki A M, Mammen P P A, Kitazume T, Peterson J A, Horton J D, Garry D J, Bianco A C, and Mangelsdorf D J. LXRs regulate the balance between fat storage and oxidation. Cell Metabolism 1: 231, 2005.
27. Kondo H, Minegishi Y, Komine Y, Mori T, Matsumoto I, Abe K, Tokimitsu I, Hase T, and Murase T. Differential regulation of intestinal lipid metabolism-related genes in obesity-resistant A/J vs. obesity-prone C57BL/6J mice. Am J Physiol Endocrinol Metab 291: E1092-1099, 2006.
28. Kota R S, Rutledge J C, Gohil K, Kumar A, Enelow R I, and Ramana C V. Regulation of gene expression in RAW 264.7 macrophage cell line by interferon-[gamma]. Biochemical and Biophysical Research Communications 342: 1137, 2006.
29. Koves T R, Li P, An J, Akimoto T, Slentz D, Ilkayeva O, Dohm G L, Yan Z, Newgard C B, and Muoio D M. Peroxisome Proliferator-activated Receptor-{gamma} Co-activator 1 {alpha}-mediated Metabolic Remodeling of Skeletal Myocytes Mimics Exercise Training and Reverses Lipid-induced Mitochondrial Inefficiency. J Biol Chem 280: 33588-33598, 2005.
30. Kreeft A J, Moen C J A, Porter G, Kasanmoentalib S, Sverdlov R, van Gorp P J, Havekes L M, Frants R R, and Hofker M H. Genomic analysis of the response of mouse models to high-fat feeding shows a major role of nuclear receptors in the simultaneous regulation of lipid and inflammatory genes. Atherosclerosis 182: 249, 2005.
31. Laaksonen D E, Niskanen L, Lakka H M, Lakka T A, and Uusitupa M. Epidemiology and treatment of the metabolic syndrome. Ann Med 36: 332-346, 2004.
32. Lee C-H, Olson P, Hevener A, Mehl I, Chong L-W, Olefsky J M, Gonzalez F J, Ham J, Kang H, Peters J M, and Evans R M. PPAR{delta} regulates glucose metabolism and insulin sensitivity. PNAS 103: 3444-3449, 2006.
33. Lee H, Braynen W, Keshav K, and Pavlidis P. ErmineJ: Tool for functional analysis of gene expression data sets. BMC Bioinformatics 6: 269, 2005.
34. Lin J, Yang R, Tarr P T, Wu P—H, Handschin C, Li S, Yang W, Pei L, Uldry M, Tontonoz P, Newgard C B, and Spiegelman B M. Hyperlipidemic Effects of Dietary Saturated Fats Mediated through PGC-1[beta] Coactivation of SREBP. Cell 120: 261, 2005.
35. Mandard S, Zandbergen F, van Straten E, Wahli W, Kuipers F, Muller M, and Kersten S. The fasting-induced adipose factor/angiopoietin-like protein 4 is physically associated with lipoproteins and governs plasma lipid levels and adiposity. J Biol Chem 281: 934-944, 2006.
36. Marguet D, Baggio L, Kobayashi T, Bernard A M, Pierres M, Nielsen P F, Ribel U, Watanabe T, Drucker D J, and Wagtmann N. Enhanced insulin secretion and improved glucose tolerance in mice lacking CD26. Proc Natl Acad Sci USA 97: 6874-6879, 2000.
37. Mitani H, Takimoto M, Hughes T E, and Kimura M. Dipeptidyl peptidase IV inhibition improves impaired glucose tolerance in high-fat diet-fed rats: study using a Fischer 344 rat substrain deficient in its enzyme activity. Jpn J Pharmacol 88: 442-450, 2002.
38. Moller D E, and Kaufman K D. METABOLIC SYNDROME: A Clinical and Molecular Perspective. Annual Review of Medicine 56: 45-62, 2005.
39. Netea M G, Joosten L A, Lewis E, Jensen D R, Voshol P J, Kullberg B J, Tack C J, van Krieken H, Kim S H, Stalenhoef A F, van de Loo F A, Verschueren I, Pulawa L, Akira S, Eckel R H, Dinarello C A, van den Berg W, and van der Meer J W. Deficiency of interleukin-18 in mice leads to hyperphagia, obesity and insulin resistance. Nat Med 12: 650-656, 2006.
40. Petit V, Arnould L, Martin P, Monnot M C, Pineau T, Besnard P, and Niot I. Chronic high-fat diet affects intestinal fat absorption and postprandial triglyceride levels in the mouse. J Lipid Res 48: 278-287, 2007.
41. Rahman S M, Dobrzyn A, Dobrzyn P, Lee S-H, Miyazaki M, and Ntambi J M. Stearoyl-CoA desaturase 1 deficiency elevates insulin-signaling components and down-regulates protein-tyrosine phosphatase 1B in muscle. PNAS 100: 11110-11115, 2003.
42. Rhee S J, Walker W A, and Cherayil B J. Developmentally Regulated Intestinal Expression of IFN-{gamma} and Its Target Genes and the Age-Specific Response to Enteric Salmonella Infection. J Immunol 175: 1127-1136, 2005.
43. Roche H M, Phillips C, and Gibney M J. The metabolic syndrome: the crossroads of diet and genetics. Proc Nutr Soc 64: 371-377, 2005.
44. Shi H, Kokoeva M V, Inouye K, Tzameli I, Yin H, and Flier J S. TLR4 links innate immunity and fatty acid-induced insulin resistance. J Clin Invest 116: 3015-3025, 2006.
45. Silha J V, Gui Y, and Murphy L J. Impaired glucose homeostasis in insulin-like growth factor-binding protein-3-transgenic mice. Am J Physiol Endocrinol Metab 283: E937-945, 2002.
46. Sparks L M, Xie H, Koza R A, Mynatt R, Hulver M W, Bray G A, and Smith S R. A High-Fat Diet Coordinately Downregulates Genes Required for Mitochondrial Oxidative Phosphorylation in Skeletal Muscle. Diabetes 54: 1926-1933, 2005.
47. Tuncman G, Hirosumi J, Solinas G, Chang L, Karin M, and Hotamisligil G S. Functional in vivo interactions between JNK1 and JNK2 isoforms in obesity and insulin resistance. PNAS 103: 10741-10746, 2006.
48. Turnbaugh P J, Ley R E, Mahowald M A, Magrini V, Mardis E R, and Gordon J I. An obesity-associated gut microbiome with increased capacity for energy harvest. Nature 444: 1027, 2006.
49. Vega R B, Huss J M, and Kelly D P. The Coactivator PGC-1 Cooperates with Peroxisome Proliferator-Activated Receptor alpha in Transcriptional Control of Nuclear Genes Encoding Mitochondrial Fatty Acid Oxidation Enzymes. Mol Cell Biol 20: 1868-1876, 2000.
50. Wang L, Liu J, Saha P, Huang J, Chan L, Spiegelman B, and Moore D D. The orphan nuclear receptor SHP regulates PGC-1[alpha] expression and energy production in brown adipocytes. Cell Metabolism 2: 227, 2005.
51. Winzell M S, and Ahren B. The high-fat diet-fed mouse: a model for studying mechanisms and treatment of impaired glucose tolerance and type 2 diabetes. Diabetes 53 Suppl 3: S215-219, 2004.

TABLE 1
Overrepresentation of GO Biological Process subsets in the small intestine
during high-fat diet intervention of C57B1/6J mice.

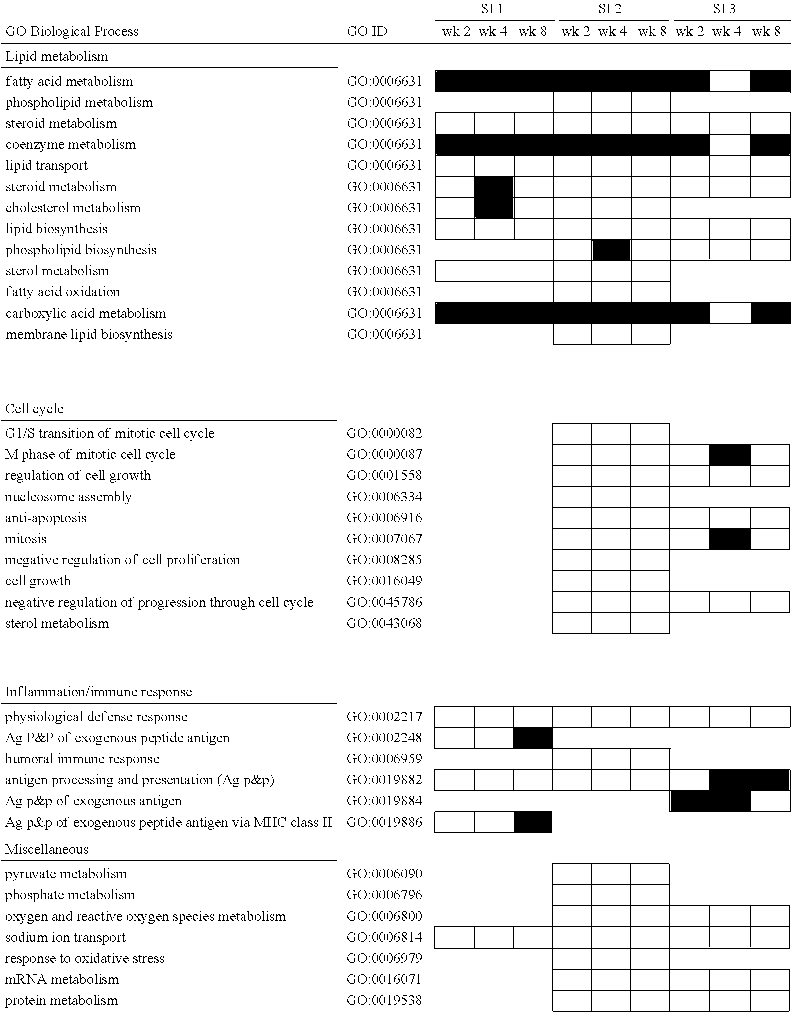

For the proximal (SI 1), middle (SI 2) and distal part of the small intestine (SI 3), GO Biological Process subsets with a FDR <0.01 and a RawScore ≧10 in at least one week of diet intervention are included. Black boxes indicate 1.0E-31 < FDR < 1.0E-08; dark grey boxes indicate 1.0E-08 < FDR < 0.01; white boxes indicate FDR > 0.01, so not significant. An empty row indicates that this part of small intestine does meet the above mentioned selection criteria.

TABLE 2
Differential gene expression of potential signaling molecules in small
intestine during high-fat diet intervention of C57B1/6J mice.

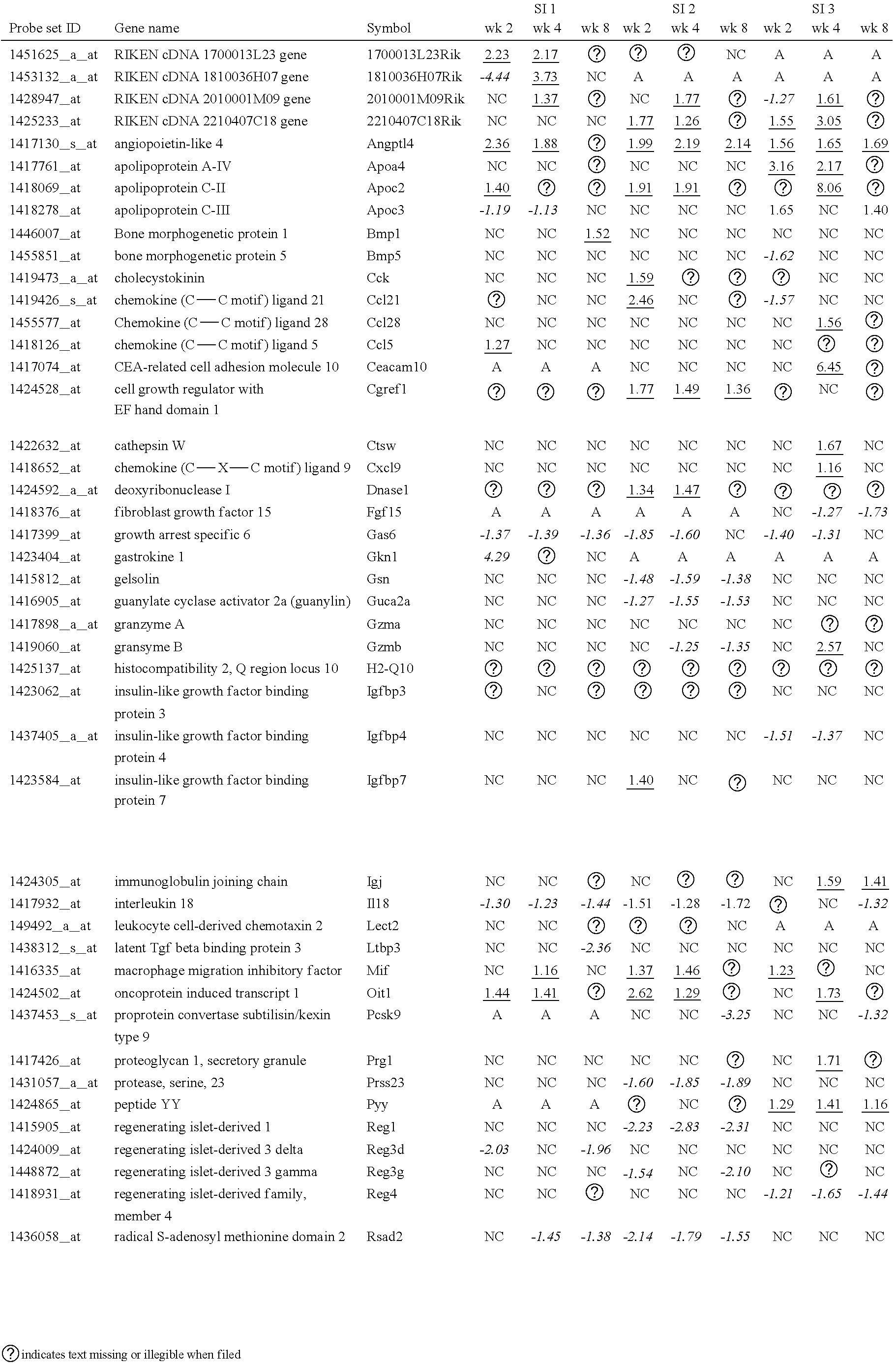

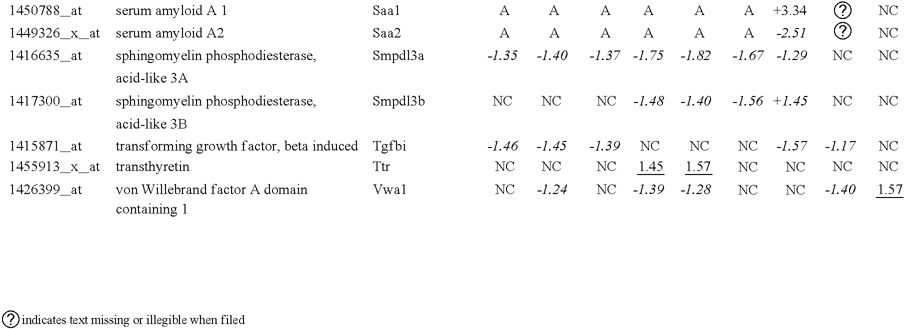

Differential gene expression, in the proximal (SI 1), middle (SI 2) and distal part of the small intestine (SI 3). Fold changes are <−1.5 and >+1.5 in at least one week of diet intervention. Underlined and italics boxes indicate significantly increased and decreased gene expression, respectively (according to Affymetrix MAS 5.0). NC = no change, A = absent.

TABLE 3
Expression of obesity-and/or insulin resistance-associated genes during high-fat
diet intervention in the small intestine of C57B1/6J mice.

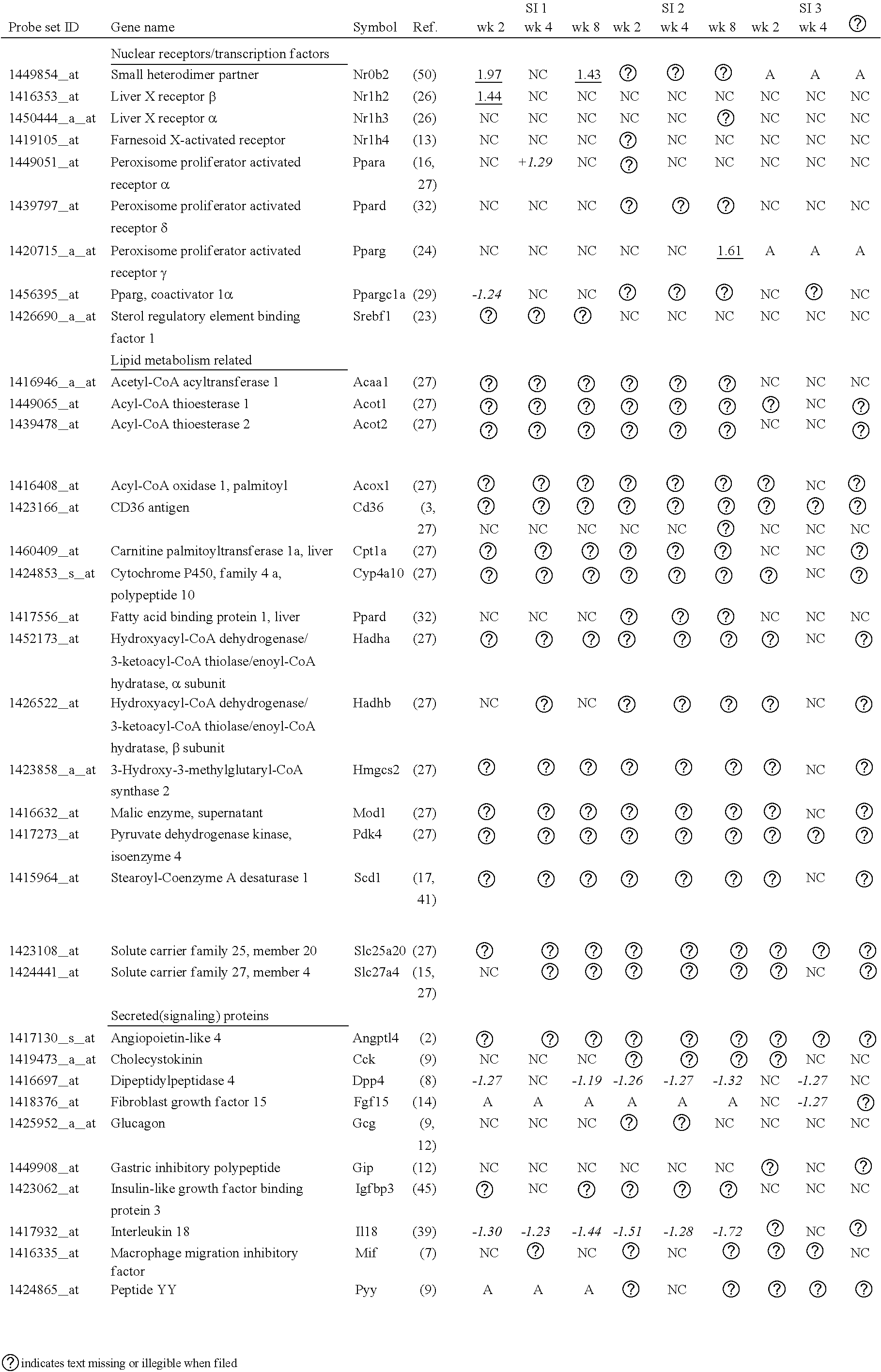

Differential gene expression of the proximal (SI 1), middle (SI 2) and distal part of the small intestine (SI 3). Underlined and italics boxes indicate significantly increased and decreased gene expression, respectively (according to Affymetrix MAS 5.0). NC = no change, A = absent.

TABLE 4
Secreted molecules in small intestine of C57B1/6J mice during HF diet
intervention.

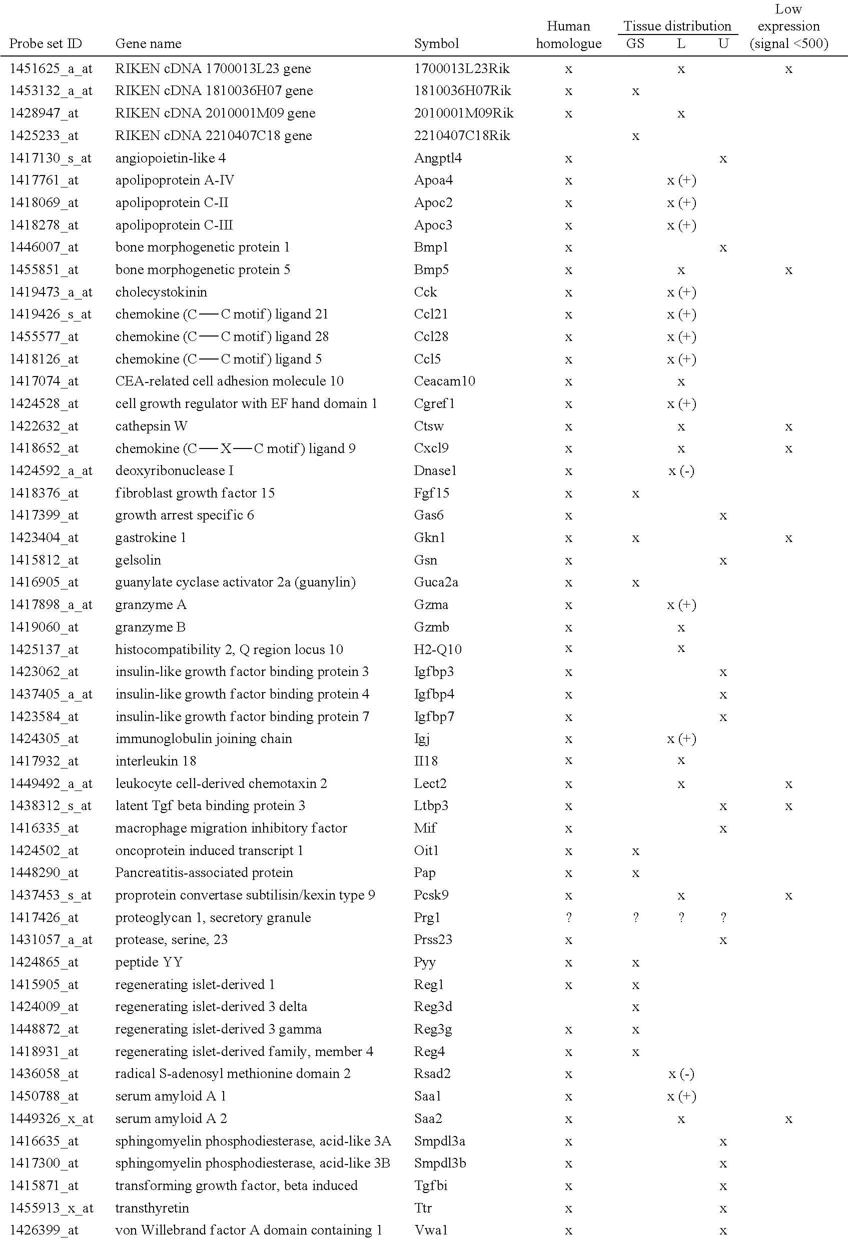

GS: gastrointestinal-restricted expression,
L: expression restricted to limited number of tissues,
U: ubiquitously expressed.
(+): high expression also in intestine,
(−): low expression in intestine.

TABLE 5

| SEQ ID NO: cDNA | | Symbol |
|---|---|---|
| (SEQ ID NO): protein | | Gene name |
| 1 | (19) | Fam3 D or Oit 1 |
| | | Oncoprotein induced transcript 1 |
| 2 | (20) | Apoa4 |
| | | Alipoprotein A-IV |
| 3 | (21) | Apoc2 |
| | | Alipoprotein C-II |
| 4 | (22) | Cck |
| | | Cholecystokinin |
| 5 | (23) | Cgref1 |
| | | Cell growth regulator with EF hand |
| | | Domain 1 |
| 6 | (24) | Fgf19 human homologue of Fgf15 |
| | | Fibroblast growth factor 15 |
| 7 | (25) | Guca2a |
| | | Guanylate cyclase activator 2a (guanylin) |
| 8 | (26) | Gzma |
| | | Granzyme A |
| 9 | (27) | HLA-G human homologueof H2-Q10 |
| | | Histocompatibility 2, Q region locus 10 |
| 10 | (28) | Igj |
| | | Immunoglobulin joining chain |
| 11, 12 | (29) | Reg3g human homologue of Pap |
| | | Pancreatitis-associated protein |
| 13 | (30) | Pyy |
| | | Peptide YY |
| 14 | (31) | Reg1a human homologue of Reg 1 |
| | | Regenerating islet-derived 1 |
| 15, 16, 17 | (32) | Reg3a human homologue of Reg 3g |
| | | Regenerating islet-derived 3 gamma |
| 18 | (33) | Reg 4 |
| | | Regenerating islet-derived family, |
| | | member 4 |

SUPPLEMENTARY TABLE S1
Diet composition

| | Low fat (LF) diet | | High fat (HF) diet | |
|---|---|---|---|---|
| | Based on formula # | | | |
| | D12450B* | | D12451* | |
| | gm % | kcal % | gm % | kcal % |
| Protein | 19 | 20 | 24 | 20 |
| Carbohydrate | 67 | 70 | 41 | 35 |
| Fat | 4 | 10 | 24 | 45 |
| Ingredients | gm | kcal | gm | kcal |
| Casein, lactic | 200 | 800 | 200 | 800 |
| L-Cystine | 3 | 12 | 3 | 12 |
| Corn Starch | 427.2 | 1709 | 72.8 | 291 |
| Maltodextrin | 100 | 400 | 100 | 400 |
| Sucrose | 172.8 | 691 | 172.8 | 691 |
| Cellulose, BW200 | 50 | 0 | 50 | 0 |
| Soybean Oil | 25 | 225 | 25 | 225 |
| Palm oil | 20 | 180 | 177.5 | 1598 |
| Mineral Mix S10026 | 10 | 0 | 10 | 0 |
| DiCalcium Phosphate | 13 | 0 | 13 | 0 |
| Calcium Carbonate | 5.5 | 0 | 5.5 | 0 |
| Potassium Citrate, 1 | 16.5 | 0 | 16.5 | 0 |
| H2O | | | | |
| Vitamin Mix V10001 | 10 | 40 | 10 | 40 |
| Choline Bitartrate | 2 | 0 | 2 | 0 |
| Total | 1055 | 4057 | 858.15 | 4057 |

*Research Diets, Inc. (New Brunswick, NJ, USA)

SUPPLEMENTARY TABLE S2
Primer sequences

| Gene symbol | Forward primer | Reverse primer |
|---|---|---|
| Abca1 | 5′-CCCAGAGCAAAAAGCGACTC-3′ | 5′-ACCATCCATGCCTACAACAAAAGG-3′ |
| Apoa4 | 5′-CAACAGGCTGAAGGCTACGAT-3′ | 5′-CGATTTTTGCGGAGACCTTGG-3′ |
| Ccnd1 | 5′-CAGAAGTGCGAAGAGGAGGTC-3′ | 5′-TCATCTTAGAGGCCACGAACAT-3′ |
| Cd36 | 5′-TCCAGCCAATGCCTTTGC-3′ | 5′-TGGAGATTACTTTTCAGTGCAGAA-3′ |
| Gsta3 | 5′-TAGAGATCGACGGGATGAAACT-3′ | 5′-CAGATCCGCCACTCCTTCT-3′ |
| Hmgcs2 | 5′-TGGTGGATGGGAAGCTGTCTA-3′ | 5′-TTCTTGCGGTAGGCTGCATAG-3′ |
| II18 | 5′-GACTCTTGCGTCAACTTCAAGG-3′ | 5′-CAGGCTGTCTTTTGTCAACGA-3′ |
| Mttp | 5′-ATACAAGCTCACGTACTCCACT-3′ | 5′-TCCACAGTAACACAACGTCCA-3′ |
| Scarb1 | 5′-TTTGGAGTGGTAGTAAAAAGGG-3′ | 5′-TGACATCAGGGACTCAGAGTAG-3′ |
| Scd1 | 5′-CCGGAGACCCTTAGATCGA-3′ | 5′-TAGCCTGTAAAAGATTTCTGCAAACC-3′ |
| Slc25a20 | 5′-CCGAAACCCATCAGTCCGTTTAA-3′ | 5′-ACATAGGTGGCTGTCCAGACAA-3′ |

SUPPLEMENTARY TABLE 3
Genes showing a consistent differential expression in the small
intestine of C57Bl/6J mice in all weeks of diet intervention.

| | | | | Fold change | | |
|---|---|---|---|---|---|---|
| | Probe set ID | Gene name | Symbol | wk 2 | wk 4 | wk 8 |
| SI 1 | 1416632_at | malic enzyme, supernatant | Mod1 | 11.24 | 8.28 | 7.01 |
| | 1425137_a_at | histocompatibility 2, Q region locus 10 | H2-Q10 | 8.11 | 9.00 | 8.46 |
| | 1436169_at | RIKEN cDNA C730029A08 gene | C730029A08Rik | 7.26 | 3.27 | 3.56 |
| | 1424853_s_at | cytochrome P450, family 4, subfamily a, polypeptide 10 | Cyp4a10 | 6.02 | 6.45 | 7.01 |
| | 1448700_at | G0/G1 switch gene 2 | G0s2 | 5.43 | 5.35 | 9.25 |
| | 1423858_a_at | 3-hydroxy-3-methylglutaryl-Coenzyme A synthase 2 | Hmgcs2 | 4.59 | 4.79 | 5.10 |
| | 1449065_at | acyl-CoA thioesterase 1 | Acot1 | 3.71 | 4.38 | 6.63 |
| | 1419622_at | UDP glucuronosyltransferase 2 family, polypeptide B5 | Ugt2b5 | 3.41 | 2.87 | 3.66 |
| | 1415964_at | stearoyl-Coenzyme A desaturase 1 | Scd1 | 3.29 | 3.25 | 1.68 |
| | 1418538_at | KDEL endoplasmic reticulum protein retention receptor | Kdelr3 | 2.95 | 4.00 | 2.99 |
| | 1424167_a_at | phosphomannomutase 1 | Pmm1 | 2.35 | 2.13 | 3.29 |
| | 1433626_at | phospholipid scramblase 4 | Plscr4 | 2.31 | 2.75 | 3.76 |
| | 1429286_at | RIKEN cDNA 1190003M12 gene | 1190003M12Rik | 1.42 | 1.79 | 5.82 |
| | 1449907_at | beta-carotene 15,15′-monooxygenase | Bcmo1 | −9.71 | −14.52 | −13.36 |
| | 1418787_at | mannose binding lectin (C) | Mbl2 | −5.50 | −16.68 | −8.11 |
| | 1421840_at | ATP-binding cassette, sub-family A, member 1 | Abca1 | −3.18 | −2.81 | −2.31 |
| | 1417651_at | cytochrome P450, family 2, subfamily c, polypeptide 29 | Cyp2c29 | −2.60 | −3.16 | −2.48 |
| | 1435370_a_at | carboxylesterase 3 | Ces3 | −2.33 | −3.81 | −2.99 |
| SI 2 | 1449065_at | acyl-CoA thioesterase 1 | Acot1 | 24.76 | 8.88 | 6.87 |
| | 1424853_s_at | cytochrome P450, family 4, subfamily a, polypeptide 10 | Cyp4a10 | 23.59 | 24.59 | 14.83 |
| | 1416632_at | malic enzyme, supernatant | Mod1 | 18.00 | 16.00 | 8.75 |
| | 1425137_a_at | histocompatibility 2, Q region locus 10 | H2-Q10 | 13.55 | 11.71 | 7.84 |
| | 1449854_at | nuclear receptor subfamily 0, group B, member 2 | Nr0b2 | 12.21 | 3.68 | 2.89 |
| | 1424266_s_at | expressed sequence AU018778 | AU018778 | 6.77 | 6.54 | 4.59 |
| | 1423858_a_at | 3-hydroxy-3-methylglutaryl-Coenzyme A synthase 2 | Hmgcs2 | 6.50 | 7.67 | 4.66 |
| | 1448700_at | G0/G1 switch gene 2 | G0s2 | 6.28 | 7.62 | 8.94 |
| | 1431688_at | hypothetical LOC73899 | LOC73899 | 5.94 | 2.01 | 4.56 |
| | 1418538_at | KDEL endoplasmic reticulum protein retention receptor | Kdelr3 | 5.58 | 4.59 | 4.20 |
| | 1421040_a_at | glutathione S-transferase, alpha 2 (Yc2) | Gsta2 | 5.10 | 3.03 | 2.57 |
| | 1419692_a_at | leukotriene C4 synthase | Ltc4s | 4.82 | 3.76 | 3.76 |
| | 1419618_at | butyrobetaine (gamma), 2-oxoglutarate dioxygenase 1 | Bbox1 | 4.76 | 5.46 | 3.89 |
| | 1427347_s_at | tubulin, beta 2 | Tubb2 | 4.69 | 4.17 | 2.69 |
| | 1417812_a_at | laminin, beta 3 | Lamb3 | 4.66 | 4.06 | 3.53 |
| | 1419622_at | UDP glucuronosyltransferase 2 family, polypeptide B5 | Ugt2b5 | 4.35 | 4.20 | 4.00 |
| | 1456558_s_at | expressed sequence C87977 | C87977 | 4.35 | 2.91 | 2.71 |
| | 1432790_at | RIKEN cDNA 9030218A15 gene | 9030218A15Rik | 4.17 | 2.10 | 1.65 |
| | 1423436_at | glutathione S-transferase, alpha 3 | Gsta3 | 4.08 | 3.46 | 3.56 |
| | 1417415_at | solute carrier family 6, member 3 | Slc6a3 | 3.68 | 5.86 | 5.17 |
| | 1418848_at | aquaporin 7 | Aqp7 | 3.66 | 3.39 | 4.32 |
| | 1415964_at | stearoyl-Coenzyme A desaturase 1 | Scd1 | 3.63 | 3.58 | 6.28 |
| | 1430780_a_at | phosphomannomutase 1 | Pmm1 | 3.63 | 4.03 | 2.11 |
| | 1452277_at | RIKEN cDNA 6330406P08 gene | 6330406P08Rik | 3.63 | 3.05 | 2.48 |
| | 1429298_at | dimethylarginine dimethylaminohydrolase 1 | Ddah1 | 3.48 | 2.45 | 1.53 |
| | 1459030_at | — | — | 3.39 | 3.51 | 3.39 |
| | 1424962_at | transmembrane 4 superfamily member 4 | Tm4sf4 | 3.36 | 3.32 | 2.51 |
| | 1420673_a_at | acyl-Coenzyme A oxidase 2, branched chain | Acox2 | 3.01 | 2.87 | 2.33 |
| | 1426452_a_at | RAB30, member RAS oncogene family | Rsb30 | 3.01 | 2.91 | 2.01 |
| | 1448777_at | minichromosome maintenance deficient 2 | Mcm2 | 2.71 | 3.05 | 2.89 |
| | 1433626_at | phospholipid scramblase 4 | Plscr4 | 2.71 | 4.08 | 5.17 |
| | 1424502_at | oncoprotein induced transcript 1 | Oit1 | 2.62 | 3.29 | 2.10 |
| | 1459059_at | RIKEN cDNA 2010308F09 gene | 2010308F09Rik | 2.11 | 5.39 | 1.28 |
| | 1449907_at | beta-carotene 15,15′-monooxygenase | Bcmo1 | −34.54 | −30.91 | −19.84 |
| | 1418787_at | mannose binding lectin (C) | Mbl2 | −14.32 | −17.88 | −8.00 |
| | 1424265_at | N-acetylneuraminate pyruvate lyase | Npl | −5.66 | −6.77 | −6.59 |
| | 1416050_a_at | scavenger receptor class B, member 1 | Scarb1 | −4.82 | −4.17 | −3.39 |
| | 1450167_at | RAB37, member of RAS oncogene family | Rab37 | −4.29 | −3.16 | −1.97 |
| | 1450392_at | ATP-binding cassette, sub-family A, member 1 | Abca1 | −3.41 | −2.60 | −1.92 |
| | 1434736_at | hepatic leukemia factor | Hlf | −3.27 | −3.14 | −2.62 |
| | 1418382_at | adenomatosis polyposis coli down-regulated 1 | Apcdd1 | −2.95 | −3.25 | −2.22 |
| | 1436021_at | RIKEN cDNA A930031D07 gene | A930031D07Rik | −2.79 | −2.45 | −3.51 |
| | 1416432_at | 6-phosphofructo-2-kinase/fructose-2,6-biphosphatase 3 | Pfkfb3 | −2.41 | −3.05 | −3.27 |
| | 1418979_at | RIKEN cDNA 9030611N15 gene | 9030611N15Rik | −2.36 | −2.35 | −2.17 |
| | 1438610_a_at | Crystallin, zeta | Cryz | −2.36 | −4.41 | −1.58 |
| | 1435370_a_at | carboxylesterase 3 | Ces3 | −2.11 | −3.01 | −2.46 |
| SI 3 | 1418069_at | apolipoprotein C-II | Apoc2 | 11.08 | 8.06 | 9.00 |
| | 1425137_a_at | histocompatibility 2, Q region locus 10 | H2-Q10 | 8.40 | 2.53 | 2.50 |
| | 1422846_at | retinol binding protein 2 | Rbp2 | 3.43 | 2.28 | 3.10 |
| | 1417761_at | apolipoprotein A-IV | Apoa4 | 3.16 | 2.17 | 3.20 |
| | 1425233_at | RIKEN cDNA 2210407C18 gene | 2210407C18Rik | 1.55 | 3.05 | 1.35 |
| | 1449907_at | beta-carotene 15,15′-monooxygenase | Bcmo1 | −3.63 | −3.39 | −2.30 |
| | 1424265_at | N-acetylneuraminate pyruvate lyase | Npl | −2.38 | −3.46 | −2.17 |
| | 1418174_at | D site albumin promoter binding protein | Dbp | −2.08 | −3.43 | −1.89 |

Consistent differential gene expression in the proximal (SI 1), middle (SI 2) and distal part of the small intestine (SI 3).
Fold changes are <−3.0 and >+3.0 in at least one week of diet intervention.

1. A method for diagnosing pre-symptomatic metabolic syndrome (PSMS) in a subject, comprising the steps of:
(a) determining the expression level of a gene in a subject which gene is represented by a polynucleotide the sequence of which is at least one of SEQ ID NO:1 to SEQ ID NO:18; and,
(b) comparing the expression level of said gene with a reference value for said expression level.

2. A method according to claim 1, wherein PSMS is diagnosed when the expression level is detected or is increased compared to said reference value.

3. A method according to claim 1, wherein the expression level of at least one of said genes is determined by measuring the quantity of encoded polypeptide and/or quantifying the amount of said polynucleotide encoding said polypeptide.

4. A method according to claim 1, wherein the expression level is determined ex vivo in a sample obtained from the subject.

5. A method according to claim 4, wherein the sample is a fluid.

6. A method according to claim 1, wherein the gene or genes is or are represented by SEQ ID NO:1 and/or SEQ ID NO:2.

7. An assay device for diagnosing PSMS in a subject, comprising a molecule which specifically binds to a polypeptide encoded by a gene represented by a nucleotide sequence of any of SEQ ID NO:1 to SEQ ID NO:18.

8. A method for diagnosing PSMS in a subject comprising assaying, in the device according to claim 7, the specific binding of said molecule to the polypeptide
wherein when presence of a polypeptide encoded by any one of polynucleotide sequences SEQ ID NO:1 to SEQ ID NO:18 is
(i) detected, or
(ii) found to exceed a reference value of said polypeptide detected by binding of said molecule to the same polypeptide in a reference sample,
PSMS is diagnosed.

9. A method for identifying a test substance that is capable of preventing, treating and/or delaying the progression of metabolic syndrome in a subject, the method comprising:
(a) contacting said test substance with a test cell population or a test animal capable of expressing a gene represented by a polynucleotide molecule the sequence of which is at least one of SEQ ID NO:1 to SEQ ID NO:18;
(b) determining the expression level of the gene or the activity or steady state level of a polypeptide encoded by said gene in the test cell population or in the test animal so contacted;
(c) comparing the expression, activity or steady state level determined in (b) with the expression, activity or steady state level of the gene or of the polypeptide in a test cell population or in a test animal that is not contacted with the substance; and,
(d) identifying a substance that produces a change in expression level, activity or steady state level of the gene or the polypeptide, when comparing the test cell population or test animal that is contacted with the substance with the test cell population or test animal that is not contacted with the substance,
thereby identifying said substance.

10. The method according to claim 9, wherein the change identified in step (d) is a decrease of the expression level of said polynucleotide.

11. The method according to claim 9, wherein the expression level is determined by quantifying the amount of encoded polypeptide and/or by quantifying the amount of the polynucleotide.

12. The method according to claim 9, wherein the test cell population comprises mammalian, cells or the test animal is a mouse.

13. A method according to claim 20, wherein the test cell population comprises colon carcinoma cell line LS174T or LOVO.

14. A substance capable of preventing, treating and/or delaying the progression of metabolic syndrome in a subject identified by the method according to claim 9.

15. The method according to claim 1 wherein said reference value is an average value for said expression level in control subjects.

16. A method according to claim 4, wherein the sample is plasma, feces, urine, blood or saliva.

17. The assay device according to claim 7 wherein the specifically binding molecule is an antibody.

18. The assay device according to claim 17 wherein the antibody is a monoclonal antibody.

19. The method according to claim 12 wherein the test cell population comprises mouse cells.

20. The method according to claim 12 wherein test cell population comprises human cells.

21. The method according to claim 12 wherein the test mouse is a C57BL/6J mouse.
